(19) United States
(12) Patent Application Publication
(10) Pub. No.: US 2015/0243897 A1
(43) Pub. Date: Aug. 27, 2015

(54) MATERIALS FOR ORGANIC ELECTROLUMINESCENCE DEVICES

(71) Applicant: Merck Patent GmbH, Darmstadt (DE)

(72) Inventors: Elvira Montenegro, Weinheim (DE); Amir H. Parham, Frankfurt Am Main (DE); Anja Jatsch, Frankfurt Am Main (DE); Christof Pflumm, Darmstadt (DE); Jones V. Kroeber, Frankfurt Am Main (DE); Thomas Eberle, Landau (DE)

(73) Assignee: Merck Patent GmbH, Darmstadt (DE)

(21) Appl. No.: 14/420,123

(22) PCT Filed: Jul. 11, 2013
(86) PCT No.: PCT/EP2013/002057

§ 371 Date: Feb. 6, 2015

(30) Foreign Application Priority Data
Aug. 10, 2012 (EP) 12005829.2

(51) Int. Cl. H01L 51/00; C07D 403/10; C07D 239/26; C07D 405/04; C07D 403/04; C07D 213/16; C07D 405/14; C07D 401/14; C07D 403/14; C07D 405/10; C07D 235/08; C07D 307/91; C07D 333/76; C07C 49/792; C07C 45/68; C09K 11/02; C07D 251/24

(52) U.S. Cl. CPC H01L 51/0056; C07D 251/24; H01L 51/0067; H01L 51/0058; H01L 51/0072; C07D 403/10; C07D 239/26; C07D 405/04; C07D 403/04; C07D 213/16; C07D 405/14; C07D 401/14; C07D 403/14; C07D 405/10; H01L 51/0071; C07D 235/08; C07D 307/91; H01L 51/0073; C07D 333/76; H01L 51/0074; C07C 49/792; C07C 45/68; C09K 11/025; H01L 51/5016

(57) ABSTRACT

The present invention relates to compounds according to formula (1) or formula (2) which are suitable for use in electronic devices, more particularly organic electroluminescence devices, and also to electronic devices which contain said compounds.

The present invention relates to materials for use in electronic devices, in particular in organic electroluminescent devices, and to electronic devices comprising these materials.

The structure of organic electroluminescent devices (OLEDs) in which organic semiconductors are employed as functional materials is described, for example, in U.S. Pat. No. 4,539,507, U.S. Pat. No. 5,151,629, EP 0676461 and WO 98/27136. The emitting materials employed here are increasingly organometallic complexes which exhibit phosphorescence instead of fluorescence (M. A. Baldo et al., Appl. Phys. Lett. 1999, 75, 4-6).

In accordance with the prior art, the matrix materials employed for phosphorescent compounds and the electron-transport materials employed are frequently heteroaromatic compounds, such as, for example, triazine derivatives or benzimidazole derivatives. Suitable matrix materials for phosphorescent compounds are also carbazole derivatives. Known for this function are, for example, spirobifluorene derivatives which are substituted by triazine groups in the 2-position, as disclosed in WO 2010/015306 and WO 2010/072300. Also known are spirobifluorene derivatives which are substituted by two triazine groups in the 4,4′-position. Both in the case of fluorescent and phosphorescent OLEDs, there continues to be a need for improvement in the case of these compounds, in particular with respect to efficiency, lifetime and operating voltage on use in an organic electroluminescent device.

The object of the present invention is the provision of compounds which are suitable for use in a fluorescent or phosphorescent OLED, in particular a phosphorescent OLED, for example as electron-transport material in an electron-transport or hole-blocking layer or as matrix material in an emitting layer.

Surprisingly, it has been found that the compounds described below achieve this object and result in significant improvements in the organic electroluminescent device, in particular with respect to the lifetime, the efficiency and the operating voltage. This applies to phosphorescent and fluorescent electroluminescent devices, in particular on use of the compounds according to the invention as electron-transport material or as matrix material. The materials generally have high thermal stability and can therefore be sublimed without decomposition and in a residue-free manner. The present invention therefore relates to these materials and to electronic devices which comprise compounds of this type.

The present invention therefore relates to a compound of the following formula (1) or (2),

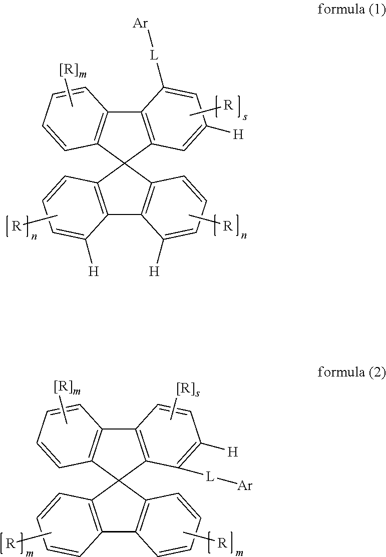

where the following applies to the symbols and indices used:
Ar is a heteroaromatic ring system having 5 to 40 aromatic ring atoms which is bonded to L via a carbon atom if L stands for a single bond, and which is bonded to L via a carbon atom or a nitrogen atom if L is not equal to a single bond, and which may be substituted by one or more radicals R¹; or Ar is an aromatic ring system having 6 to 40 aromatic ring atoms, which may be substituted by one or more radicals R¹, if L stands for C(═O);
L is a single bond, C(═O) or an aromatic ring system having 5 to 24 aromatic ring atoms, which may be substituted by one or more radicals R;
R, R¹ is selected on each occurrence, identically or differently, from the group consisting of H, D, F, Cl, Br, I, CN, Si(R²)₃, a straight-chain alkyl, alkoxy or thioalkyl group having 1 to 40 C atoms or a branched or cyclic alkyl, alkoxy or thioalkyl group having 3 to 40 C atoms, each of which may be substituted by one or more radicals R², where in each case one or more non-adjacent CH₂ groups may be replaced by Si(R²)₂, C═NR², P(═O)(R²), SO, SO₂, NR², O, S or CONR² and where one or more H atoms may be replaced by D, F, Cl, Br or I, an aromatic or heteroaromatic ring system having 6 to 40 C atoms, which may in each case be substituted by one or more radicals R², an aryloxy group having 5 to 60 aromatic ring atoms, which may be substituted by one or more radicals R², or an aralkyl group having 5 to 60 aromatic ring atoms, which may in each case be substituted by one or more radicals R², where two or more adjacent substituents R or R¹ may optionally form a mono- or polycyclic, aliphatic ring system, which may be substituted by one or more radicals R²;
R² is selected from the group consisting of H, D, F, an aliphatic hydrocarbon radical having 1 to 20 C atoms or an aromatic or heteroaromatic ring system having 5 to 30 C atoms, in which one or more H atoms may be replaced by D or F, where two or more adjacent substituents R² may form a mono- or polycyclic, aliphatic, ring system with one another;
s is 0, 1 or 2;
m is on each occurrence, identically or differently, 0, 1, 2, 3 or 4;
n is on each occurrence, identically or differently, 0, 1, 2 or 3.

An aryl group in the sense of this invention contains 6 to 24 C atoms; a heteroaryl group in the sense of this invention contains 2 to 24 C atoms and at least one heteroatom, with the proviso that the sum of C atoms and heteroatoms is at least 5. The heteroatoms are preferably selected from N, and/or S. An aryl group or heteroaryl group here is taken to mean either a simple aromatic ring, i.e. benzene, or a simple heteroaromatic ring, for example pyridine, pyrimidine, thiophene, etc., or a condensed aryl or heteroaryl group, for example naphthalene, anthracene, phenanthrene, quinoline, isoquinoline, etc.

An aromatic ring system in the sense of this invention contains 6 to 40 C atoms in the ring system. A heteroaromatic ring system in the sense of this invention contains 1 to 40 C atoms and at least one heteroatom in the ring system, with the proviso that the sum of C atoms and heteroatoms is at least 5. The heteroatoms are preferably selected from N, O and/or S. An aromatic or heteroaromatic ring system in the sense of this invention is intended to be taken to mean a system which does not necessarily contain only aryl or heteroaryl groups, but instead in which, in addition, a plurality of aryl or heteroaryl groups may be interrupted by a non-aromatic unit (preferably less than 10% of the atoms other than H), such as, for example, a C, N or O atom or a carbonyl group. Thus, for example, systems such as 9,9′-spirobifluorene, 9,9-diaryffluorene, triarylamine, diaryl ether, stilbene, etc., are also intended to be taken to be aromatic ring systems in the sense of this invention, as are systems in which two or more aryl groups are interrupted, for example, by a linear or cyclic alkyl group or by a silyl group. Furthermore, systems in which two or more aryl or heteroaryl groups are bonded directly to one another, such as, for example, biphenyl, terphenyl or quaterphenyl, are likewise intended to be taken to be an aromatic or heteroaromatic ring system.

A cyclic alkyl, alkoxy or thioalkoxy group in the sense of this invention is taken to mean a monocyclic, bicyclic or polycyclic group.

For the purposes of the present invention, a C₁- to C₄₀-alkyl group, in which, in addition, individual H atoms or CH₂ groups may be substituted by the above-mentioned groups, is taken to mean, for example, the radicals methyl, ethyl, n-propyl, i-propyl, cyclopropyl, n-butyl, i-butyl, s-butyl, t-butyl, cyclobutyl, 2-methylbutyl, n-pentyl, s-pentyl, tert-pentyl, 2-pentyl, neopentyl, cyclopentyl, n-hexyl, s-hexyl, tert-hexyl, 2-hexyl, 3-hexyl, neohexyl, cyclohexyl, 1-methylcyclopentyl, 2-methylpentyl, n-heptyl, 2-heptyl, 3-heptyl, 4-heptyl, cycloheptyl, 1-methylcyclohexyl, n-octyl, 2-ethylhexyl, cyclooctyl, 1-bicyclo[2.2.2]octyl, 2-bicyclo[2.2.2]octyl, 2-(2,6-dimethyl)octyl, 3-(3,7-dimethyl)octyl, adamantyl, trifluoromethyl, pentafluoroethyl, 2,2,2-trifluoroethyl, 1,1-dimethyl-n-hex-1-yl-, 1,1-dimethyl-n-hept-1-yl-, 1,1-dimethyl-n-oct-1-yl-, 1,1-dimethyl-n-dec-1-yl-, 1,1-dimethyl-n-dodec-1-yl-, 1,1-dimethyl-n-tetradec-1-yl-, 1,1-dimethyl-n-hexadec-1-yl-, 1,1-dimethyl-n-octadec-1-yl-, 1,1-diethyl-n-hex-1-yl-, 1,1-diethyl-n-hept-1-yl-, 1,1-diethyl-n-oct-1-yl-, 1,1-diethyl-n-dec-1-yl-, 1,1-diethyl-n-dodec-1-yl-, 1,1-diethyl-n-tetradec-1-yl-, 1,1-diethyln-n-hexadec-1-yl-, 1,1-diethyl-n-octadec-1-yl-, 1-(n-propyl)-cyclohex-1-yl-, 1-(n-butyl)-cyclohex-1-yl-, 1-(n-hexyl)cyclohex-1-yl-, 1-(n-octyl)-cyclohex-1-yl- and 1-(n-decyl)cyclohex-1-yl-. An alkenyl group is taken to mean, for example, ethenyl, propenyl, butenyl, pentenyl, cyclopentenyl, hexenyl, cyclohexenyl, heptenyl, cycloheptenyl, octenyl, cyclooctenyl or cyclooctadienyl. An alkynyl group is taken to mean, for example, ethynyl, propynyl, butynyl, pentynyl, hexynyl, heptynyl or octynyl. A C₁- to C₄₀-alkoxy group is taken to mean, for example, methoxy, trifluoromethoxy, ethoxy, n-propoxy, i-propoxy, n-butoxy, i-butoxy, s-butoxy, t-butoxy or 2-methylbutoxy.

An aromatic or heteroaromatic ring system having 5-60 aromatic ring atoms, which may also in each case be substituted by the radicals mentioned above and which may be linked to the aromatic or heteroaromatic ring system via any desired positions, is taken to mean, for example, groups derived from benzene, naphthalene, anthracene, benzanthracene, phenanthrene, benzophenanthrene, pyrene, chrysene, perylene, fluoranthene, benzofluoranthene, naphthacene, pentacene, benzopyrene, biphenyl, biphenylene, terphenyl, terphenylene, fluorene, spirobifluorene, dihydrophenanthrene, dihydropyrene, tetrahydropyrene, cis- or trans-indenofluorene, cis- or trans-monobenzoindenofluorene, cis- or trans-dibenzoindenofluorene, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, indolocarbazole, indenocarbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubin, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

In a preferred embodiment of the invention, the compounds of the formula (1) are selected from the compounds of the following formula (1a) and the compounds of the formula (2) are selected from the compounds of the following formula (2a),

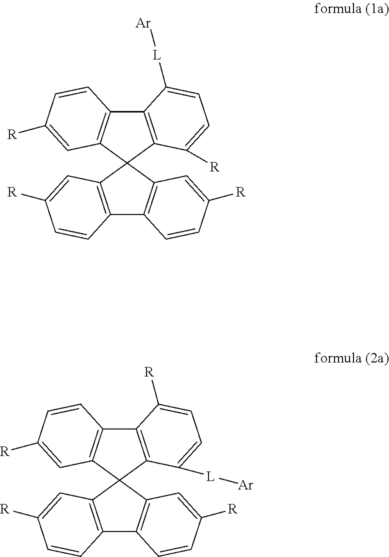

where the symbols used have the meanings given above.

Particular preference is given to the compounds of the following formula (1 b) and (2b),

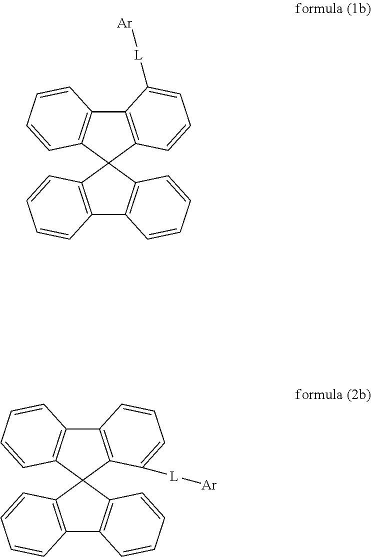

where the symbols used have the meanings given above.

Very particular preference is given to the compounds of the formula (1) or (1a) or (1 b) in which the group -L-Ar is bonded in the 4-position of the spirobifluorene.

In a preferred embodiment of the invention, L is a single bond, C(═O) or an aromatic ring system having 6 to 12 aromatic ring atoms, which may be substituted by one or more radicals R. L is particularly preferably a single bond or an ortho-, meta- or para-linked phenylene group, which may be substituted by one or more radicals R, but is preferably unsubstituted.

In a further preferred embodiment of the invention, Ar is a heteroaromatic ring system having 5 to 24 aromatic ring atoms, in particular having 5 to 13 aromatic ring atoms, which may in each case be substituted by one or more radicals R¹. The group Ar is bonded to L via a carbon atom if L stands for a single bond. Furthermore, it may also be bonded to L via a nitrogen atom if L is not equal to a single bond, for example a carbazole group, indolocarbazole group or indenocarbazole group bonded via the nitrogen atom. Ar is furthermore preferably an aromatic ring system having 6 to 24 aromatic ring atoms, which may be substituted by one or more radicals R¹, if L stands for C(═O).

Particularly preferred groups Ar are selected from the group consisting of triazine, pyrimidine, pyrazine, pyridazine, pyridine, pyrazole, imidazole, oxazole, oxadiazole, thiazole, benzimidazole, benzofuran, benzothiophene, indole, dibenzofuran, dibenzothiophene, carbazole, indenocarbazole and indolocarbazole, where these groups may each be substituted by one or more radicals R¹.

In a particularly preferred embodiment of the invention, the group Ar is selected from the structures of the following formulae (Ar-1) to (Ar-24),

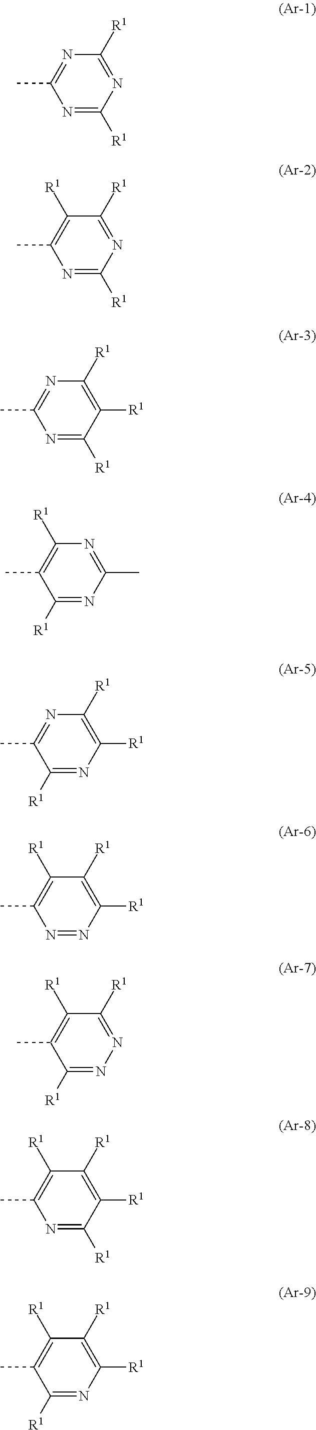

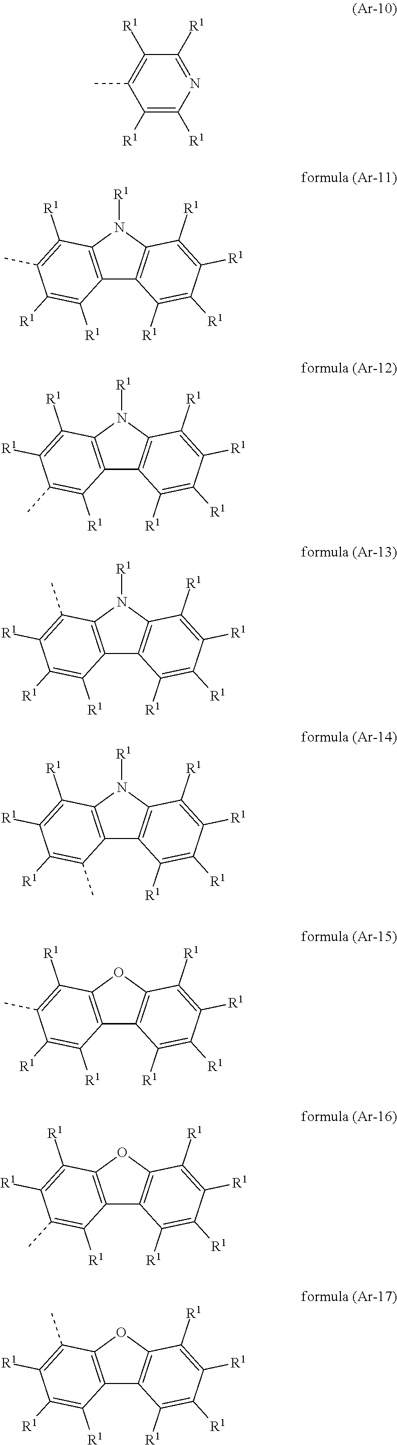

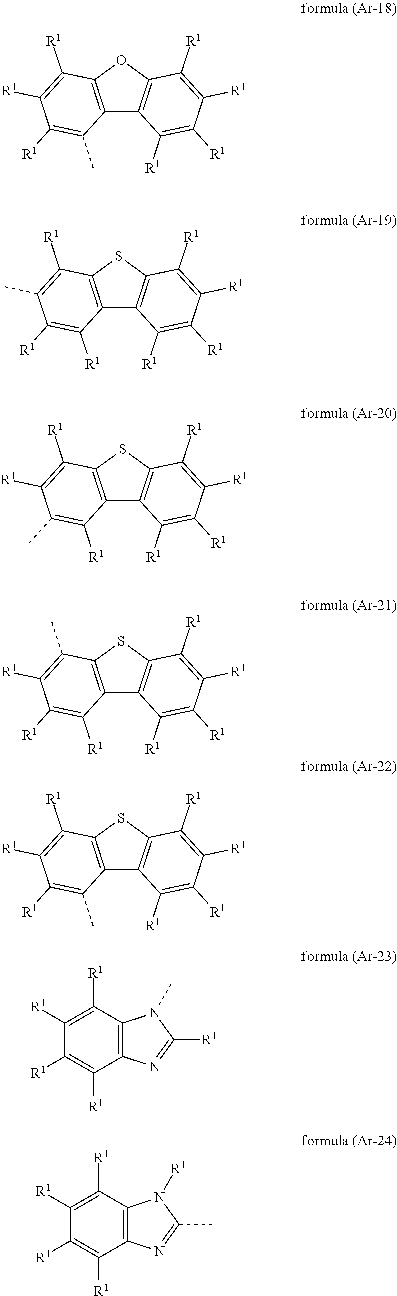

where the dashed bond indicates the bond to L and R¹ has the meanings given above.

Furthermore, the radical R¹ which is bonded to the nitrogen atom in the groups (Ar-11) to (Ar-14) and (Ar-24) preferably stands for a phenyl group, which may be substituted by one or more radicals R².

Ar is particularly preferably a triazine group, i.e. a group of the above-mentioned formula (Ar-1).

If L stands for C(═O), Ar furthermore preferably stands for an aromatic ring system selected from the group consisting of phenyl, biphenyl, ortho-, meta- or para-terphenyl, ortho-, meta-, para- or branched quaterphenyl, 1-, 2-, 3- or 4-fluorenyl or 1-, 2-, 3- or 4-spirobifluorenyl, each of which may be substituted by one or more radicals R¹.

In a preferred embodiment of the invention, R or R¹ is selected, identically or differently on each occurrence, from the group consisting of H, D, F, CN, a straight-chain alkyl or alkoxy group having 1 to 10 C atoms or a branched or cyclic alkyl or alkoxy group having 3 to 10 C atoms, each of which may be substituted by one or more radicals R², where one or more non-adjacent CH₂ groups may be replaced by O and where one or more H atoms may be replaced by F, an aromatic or heteroaromatic ring system having 6 to 24 aromatic ring atoms, which may in each case be substituted by one or more radicals R².

In a particularly preferred embodiment of the invention, R or R¹ is selected on each occurrence, identically or differently, from the group consisting of H, F, a straight-chain alkyl group having 1 to 5 C atoms or a branched or cyclic alkyl group having 3 to 6 C atoms, an aromatic or heteroaromatic ring system having 5 to 18 aromatic ring atoms, which may in each case be substituted by one or more radicals R².

The radicals R, R¹ and R² here preferably contain no condensed aryl or heteroaryl groups in which more than two aromatic or heteroaromatic six-membered rings are condensed directly onto one another, i.e., for example, no anthracene or pyrene groups. The radicals R, R¹ and R² particularly preferably contain absolutely no condensed aryl or heteroaryl groups in which aromatic or heteroaromatic six-membered rings are condensed directly onto one another, i.e. also, for example, no naphthalene groups.

For compounds which are processed by vacuum evaporation, the alkyl groups here preferably have not more than four C atoms, particularly preferably not more than 1 C atom. For compounds which are processed from solution, compounds which are substituted by linear, branched or cyclic alkyl groups having up to 10 C atoms or which are substituted by oligoarylene groups, for example ortho-, meta-, para- or branched terphenyl or quaterphenyl groups, are also suitable.

In a preferred embodiment of the invention, R² is selected, identically or differently on each occurrence, from the group consisting of H, a straight-chain alkyl group having 1 to 10 C atoms or a branched or cyclic alkyl group having 3 to 10 C atoms or an aromatic ring system having 6 to 24 C atoms. R² is particularly preferably, identically or differently on each occurrence, H or a methyl group, very particularly preferably H.

Particular preference is given to compounds of the formulae (1), (1a), (1b) or (2), (2a) and (2b) in which the preferred embodiments mentioned above occur simultaneously. Particular preference is therefore given to compounds for which:
L is a single bond, C(═O) or an aromatic ring system having 6 to 12 aromatic ring atoms, which may be substituted by one or more radicals R;
Ar is a heteroaromatic ring system having 5 to 13 aromatic ring atoms, which may be substituted by one or more radicals R¹, where Ar is bonded to L via a carbon atom if L stands for a single bond, or is bonded to L via a carbon or nitrogen atom if L is not equal to a single bond; or is an aromatic ring system having 6 to 24 aromatic ring atoms, which may be substituted by one or more radicals R¹, if L stands for C(═O);
R, R¹ is selected, identically or differently on each occurrence, from the group consisting of H, D, F, CN, a straight-chain alkyl or alkoxy group having 1 to 10 C atoms or a branched or cyclic alkyl or alkoxy group having 3 to 10 C atoms, each of which may be substituted by one or more radicals R², where one or more non-adjacent CH₂ groups may be replaced by 0 and where one or more H atoms may be replaced by F, an aromatic or heteroaromatic ring system having 6 to 24 aromatic ring atoms, which may in each case be substituted by one or more radicals R².

Very particular preference is given to compounds of the formulae (1), (1a), (1 b) or (2), (2a) and (2b) for which:
L is a single bond or an ortho-, meta- or para-linked phenylene group, which may be substituted by one or more radicals R, but is preferably unsubstituted;
Ar is selected from the group consisting of triazine, pyrimidine, pyrazine, pyridazine, pyridine, pyrazole, imidazole, oxazole, oxadiazole, thiazole, benzimidazole, benzofuran, benzothiophene, indole, dibenzofuran, dibenzothiophene, carbazole, indenocarbazole and indolocarbazole, where these groups may each be substituted by one or more radicals R¹, preferably groups of the formulae (Ar-1) to (Ar-22);
R, R¹ is selected on each occurrence, identically or differently, from the group consisting of H, F, a straight-chain alkyl group having 1 to 5 C atoms or a branched or cyclic alkyl group having 3 to 6 C atoms, an aromatic or heteroaromatic ring system having 5 to 18 aromatic ring atoms, which may in each case be substituted by one or more radicals R².

Examples of suitable compounds according to the invention are the compounds shown in the following table:

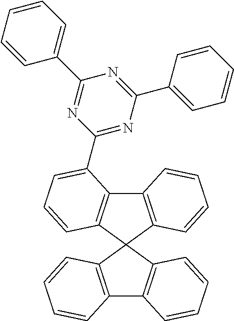 [SMILES: c1ccc(-c2nc(-c3ccccc3)nc(-c3cccc4c3-c3ccccc3C43c4ccccc4-c4ccccc43)n2)cc1]

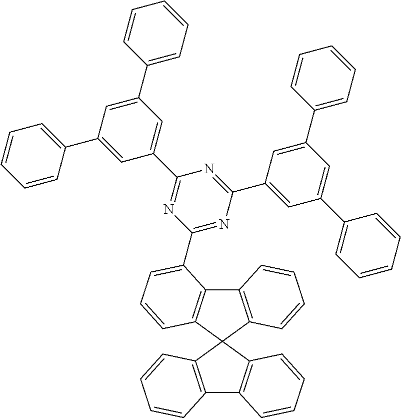 [SMILES: c1ccc(-c2cc(-c3ccccc3)cc(-c3nc(-c4cc(-c5ccccc5)cc(-c5ccccc5)c4)nc(-c4cccc5c4-c4ccccc4C54c5ccccc5-c5ccccc54)n3)c2)cc1]

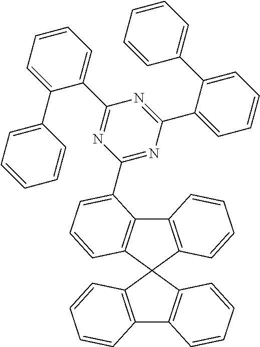 [SMILES: c1ccc(-c2ccccc2-c2nc(-c3ccccc3-c3ccccc3)nc(-c3cccc4c3-c3ccccc3C43c4ccccc4-c4ccccc43)n2)cc1]

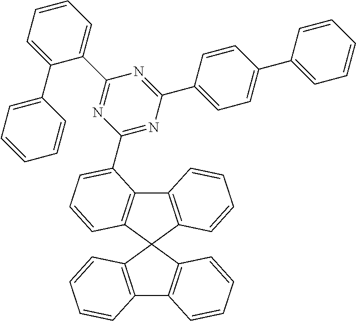 [SMILES: c1ccc(-c2ccc(-c3nc(-c4ccccc4-c4ccccc4)nc(-c4cccc5c4-c4ccccc4C54c5ccccc5-c5ccccc54)n3)cc2)cc1]

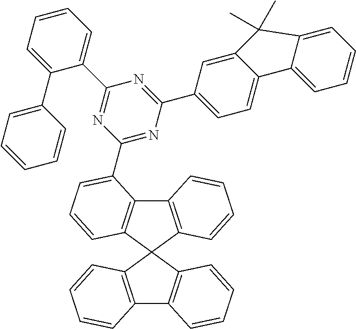 [SMILES: CC1(C)c2ccccc2-c2ccc(-c3nc(-c4ccccc4-c4ccccc4)nc(-c4cccc5c4-c4ccccc4C54c5ccccc5-c5ccccc54)n3)cc21]

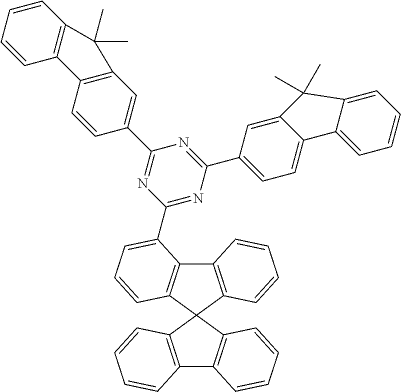 [SMILES: CC1(C)c2ccccc2-c2ccc(-c3nc(-c4ccc5c(c4)C(C)(C)c4ccccc4-5)nc(-c4cccc5c4-c4ccccc4C54c5ccccc5-c5ccccc54)n3)cc21]

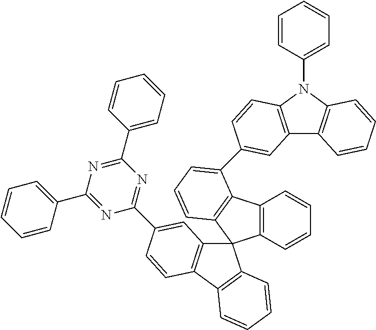 [SMILES: c1ccc(-c2nc(-c3ccccc3)nc(-c3ccc4c(c3)C3(c5ccccc5-4)c4ccccc4-c4c(-c5ccc6c(c5)c5ccccc5n6-c5ccccc5)cccc43)n2)cc1]

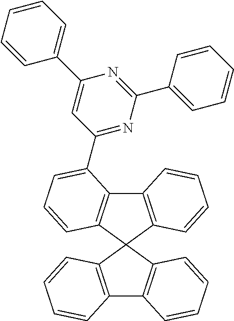 [SMILES: c1ccc(-c2cc(-c3cccc4c3-c3ccccc3C43c4ccccc4-c4ccccc43)nc(-c3ccccc3)n2)cc1]

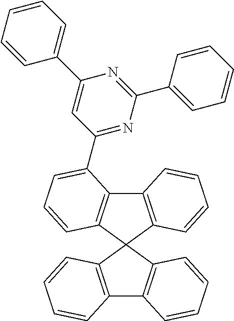 [SMILES: c1ccc(-c2cc(-c3cccc4c3-c3ccccc3C43c4ccccc4-c4ccccc43)nc(-c3ccccc3)n2)cc1]

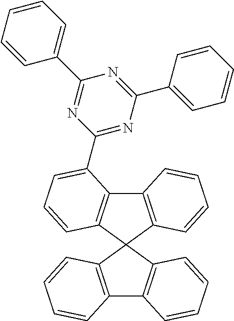 [SMILES: c1ccc(-c2nc(-c3ccccc3)nc(-c3cccc4c3-c3ccccc3C43c4ccccc4-c4ccccc43)n2)cc1]

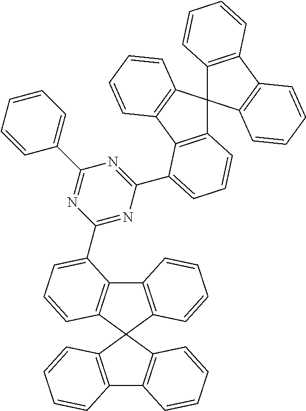 [SMILES: c1ccc(-c2nc(-c3cccc4c3-c3ccccc3C43c4ccccc4-c4ccccc43)nc(-c3cccc4c3-c3ccccc3C43c4ccccc4-c4ccccc43)n2)cc1]

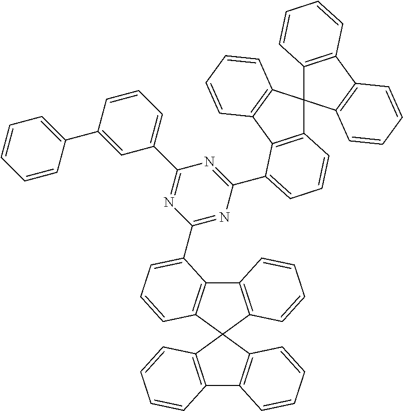 [SMILES: c1ccc(-c2cccc(-c3nc(-c4cccc5c4-c4ccccc4C54c5ccccc5-c5ccccc54)nc(-c4cccc5c4-c4ccccc4C54c5ccccc5-c5ccccc54)n3)c2)cc1]

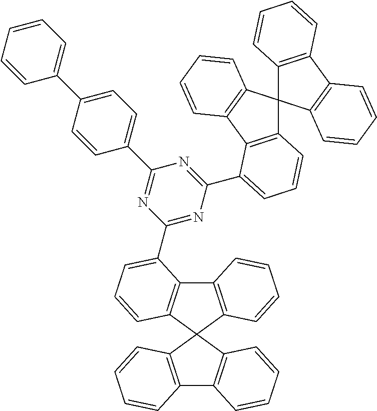 [SMILES: c1ccc(-c2ccc(-c3nc(-c4cccc5c4-c4ccccc4C54c5ccccc5-c5ccccc54)nc(-c4cccc5c4-c4ccccc4C54c5ccccc5-c5ccccc54)n3)cc2)cc1]

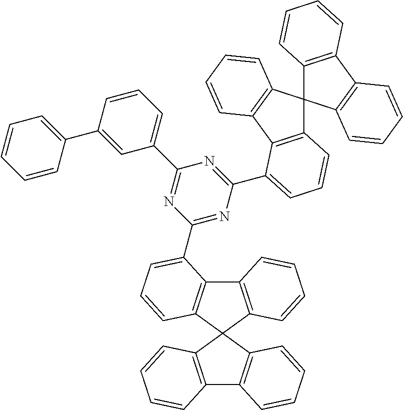 [SMILES: c1ccc(-c2cccc(-c3nc(-c4cccc5c4-c4ccccc4C54c5ccccc5-c5ccccc54)nc(-c4cccc5c4-c4ccccc4C54c5ccccc5-c5ccccc54)n3)c2)cc1]

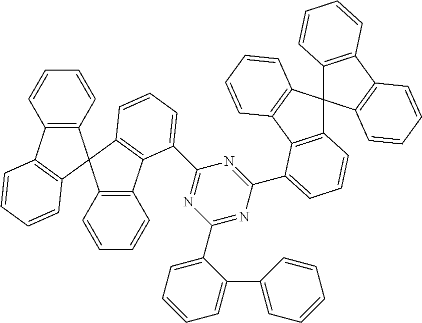 [SMILES: c1ccc(-c2ccccc2-c2nc(-c3cccc4c3-c3ccccc3C43c4ccccc4-c4ccccc43)nc(-c3cccc4c3-c3ccccc3C43c4ccccc4-c4ccccc43)n2)cc1]

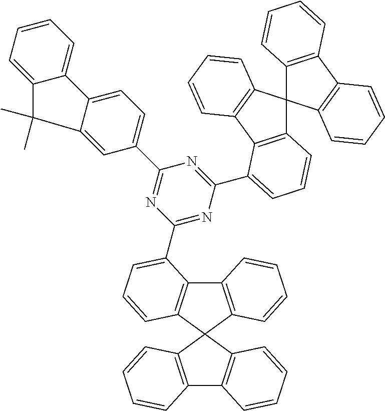 [SMILES: CC1(C)c2ccccc2-c2ccc(-c3nc(-c4cccc5c4-c4ccccc4C54c5ccccc5-c5ccccc54)nc(-c4cccc5c4-c4ccccc4C54c5ccccc5-c5ccccc54)n3)cc21]

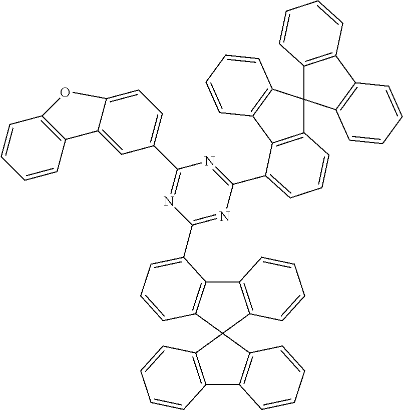 [SMILES: c1ccc2c(c1)-c1ccccc1C21c2ccccc2-c2c(-c3nc(-c4ccc5oc6ccccc6c5c4)nc(-c4cccc5c4-c4ccccc4C54c5ccccc5-c5ccccc54)n3)cccc21]

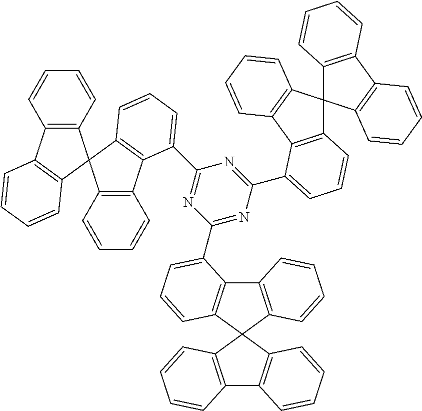 [SMILES: c1ccc2c(c1)-c1ccccc1C21c2ccccc2-c2c(-c3nc(-c4cccc5c4-c4ccccc4C54c5ccccc5-c5ccccc54)nc(-c4cccc5c4-c4ccccc4C54c5ccccc5-c5ccccc54)n3)cccc21]

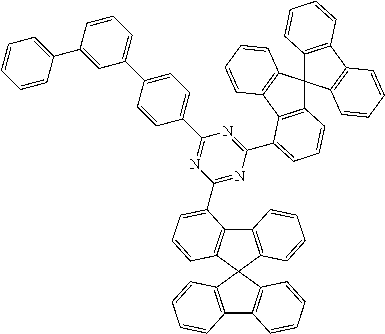 [SMILES: c1ccc(-c2cccc(-c3ccc(-c4nc(-c5cccc6c5-c5ccccc5C65c6ccccc6-c6ccccc65)nc(-c5cccc6c5-c5ccccc5C65c6ccccc6-c6ccccc65)n4)cc3)c2)cc1]

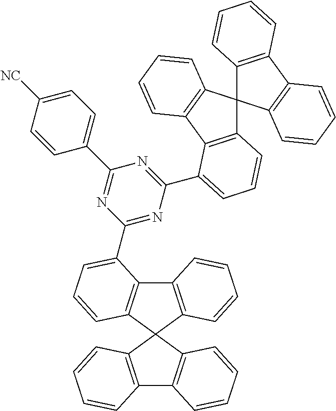 [SMILES: N#Cc1ccc(-c2nc(-c3cccc4c3-c3ccccc3C43c4ccccc4-c4ccccc43)nc(-c3cccc4c3-c3ccccc3C43c4ccccc4-c4ccccc43)n2)cc1]

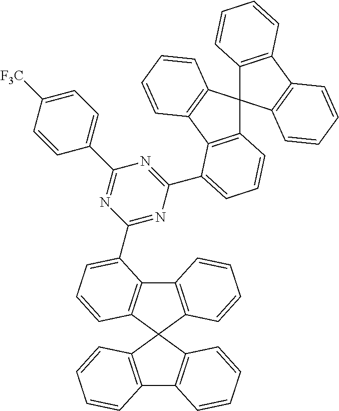 [SMILES: FC(F)(F)c1ccc(-c2nc(-c3cccc4c3-c3ccccc3C43c4ccccc4-c4ccccc43)nc(-c3cccc4c3-c3ccccc3C43c4ccccc4-c4ccccc43)n2)cc1]

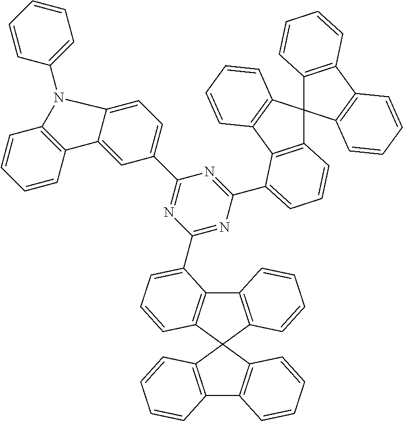 [SMILES: c1ccc(-n2c3ccccc3c3cc(-c4nc(-c5cccc6c5-c5ccccc5C65c6ccccc6-c6ccccc65)nc(-c5cccc6c5-c5ccccc5C65c6ccccc6-c6ccccc65)n4)ccc32)cc1]

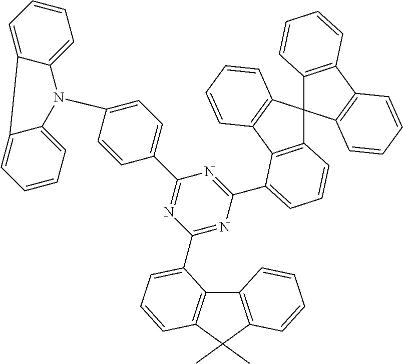 [SMILES: CC1(C)c2ccccc2-c2c(-c3nc(-c4ccc(-n5c6ccccc6c6ccccc65)cc4)nc(-c4cccc5c4-c4ccccc4C54c5ccccc5-c5ccccc54)n3)cccc21]

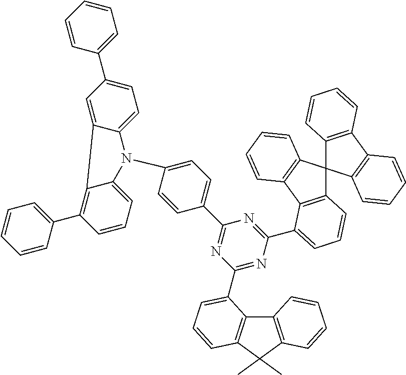 [SMILES: CC1(C)c2ccccc2-c2c(-c3nc(-c4ccc(-n5c6ccc(-c7ccccc7)cc6c6c(-c7ccccc7)cccc65)cc4)nc(-c4cccc5c4-c4ccccc4C54c5ccccc5-c5ccccc54)n3)cccc21]

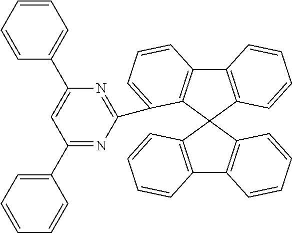 [SMILES: c1ccc(-c2cc(-c3ccccc3)nc(-c3cccc4c3C3(c5ccccc5-c5ccccc53)c3ccccc3-4)n2)cc1]

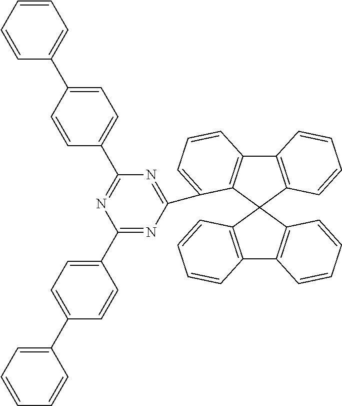 [SMILES: c1ccc(-c2ccc(-c3nc(-c4ccc(-c5ccccc5)cc4)nc(-c4cccc5c4C4(c6ccccc6-c6ccccc64)c4ccccc4-5)n3)cc2)cc1]

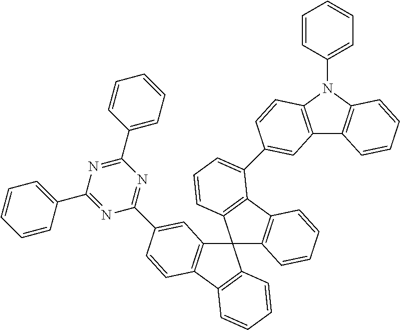 [SMILES: c1ccc(-c2nc(-c3ccccc3)nc(-c3ccc4c(c3)C3(c5ccccc5-4)c4ccccc4-c4c(-c5ccc6c(c5)c5ccccc5n6-c5ccccc5)cccc43)n2)cc1]

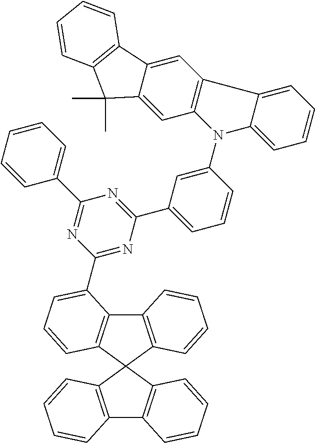 [SMILES: CC1(C)c2ccccc2-c2cc3c4ccccc4n(-c4cccc(-c5nc(-c6ccccc6)nc(-c6cccc7c6-c6ccccc6C76c7ccccc7-c7ccccc76)n5)c4)c3cc21]

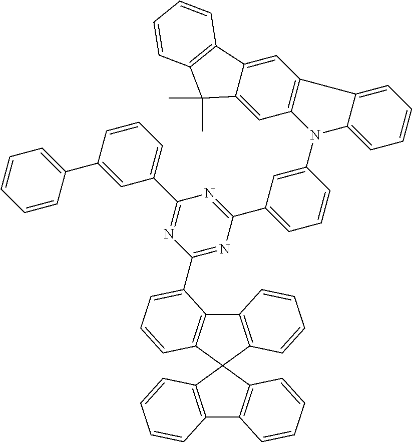 [SMILES: CC1(C)c2ccccc2-c2cc3c4ccccc4n(-c4cccc(-c5nc(-c6cccc(-c7ccccc7)c6)nc(-c6cccc7c6-c6ccccc6C76c7ccccc7-c7ccccc76)n5)c4)c3cc21]

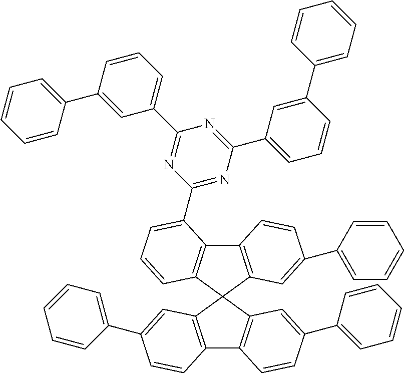 [SMILES: c1ccc(-c2cccc(-c3nc(-c4cccc(-c5ccccc5)c4)nc(-c4cccc5c4-c4ccc(-c6ccccc6)cc4C54c5cc(-c6ccccc6)ccc5-c5ccc(-c6ccccc6)cc54)n3)c2)cc1]

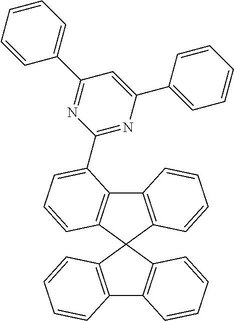 [SMILES: c1ccc(-c2cc(-c3ccccc3)nc(-c3cccc4c3-c3ccccc3C43c4ccccc4-c4ccccc43)n2)cc1]

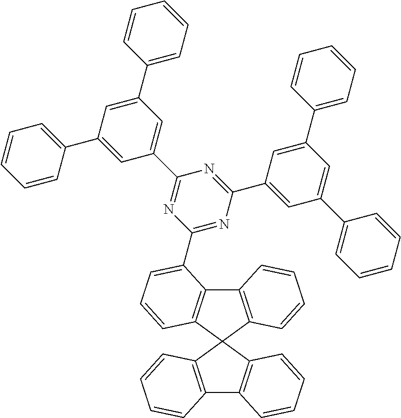 [SMILES: c1ccc(-c2cc(-c3ccccc3)cc(-c3nc(-c4cc(-c5ccccc5)cc(-c5ccccc5)c4)nc(-c4cccc5c4-c4ccccc4C54c5ccccc5-c5ccccc54)n3)c2)cc1]

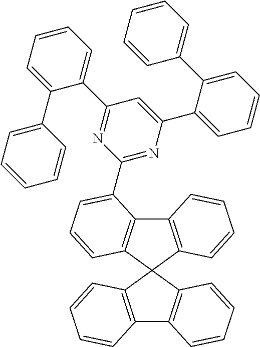 [SMILES: c1ccc(-c2ccccc2-c2cc(-c3ccccc3-c3ccccc3)nc(-c3cccc4c3-c3ccccc3C43c4ccccc4-c4ccccc43)n2)cc1]

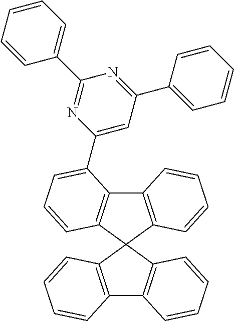 [SMILES: c1ccc(-c2cc(-c3cccc4c3-c3ccccc3C43c4ccccc4-c4ccccc43)nc(-c3ccccc3)n2)cc1]

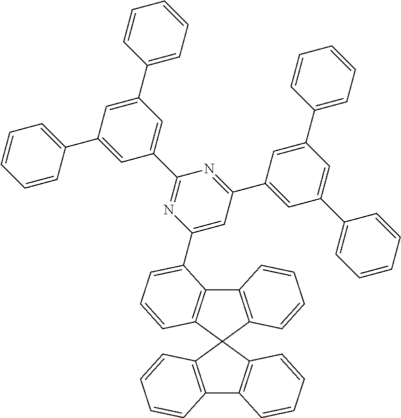 [SMILES: c1ccc(-c2cc(-c3ccccc3)cc(-c3cc(-c4cccc5c4-c4ccccc4C54c5ccccc5-c5ccccc54)nc(-c4cc(-c5ccccc5)cc(-c5ccccc5)c4)n3)c2)cc1]

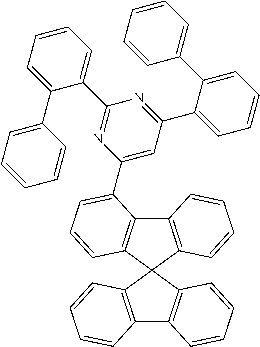 [SMILES: c1ccc(-c2ccccc2-c2cc(-c3cccc4c3-c3ccccc3C43c4ccccc4-c4ccccc43)nc(-c3ccccc3-c3ccccc3)n2)cc1]

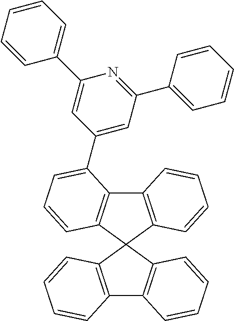 [SMILES: c1ccc(-c2cc(-c3cccc4c3-c3ccccc3C43c4ccccc4-c4ccccc43)cc(-c3ccccc3)n2)cc1]

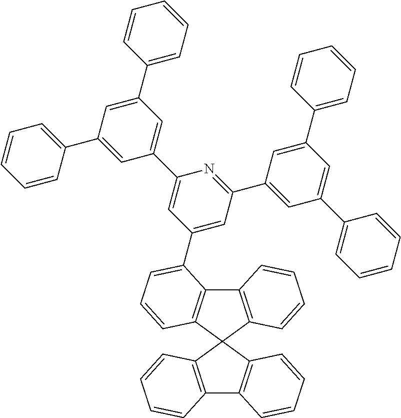 [SMILES: c1ccc(-c2cc(-c3ccccc3)cc(-c3cc(-c4cccc5c4-c4ccccc4C54c5ccccc5-c5ccccc54)cc(-c4cc(-c5ccccc5)cc(-c5ccccc5)c4)n3)c2)cc1]

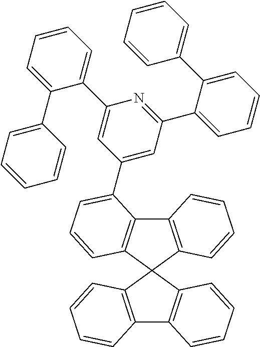 [SMILES: c1ccc(-c2ccccc2-c2cc(-c3cccc4c3-c3ccccc3C43c4ccccc4-c4ccccc43)cc(-c3ccccc3-c3ccccc3)n2)cc1]

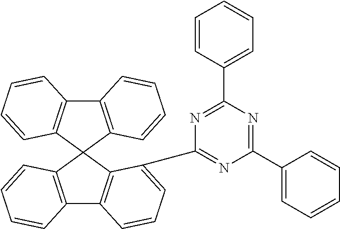 [SMILES: c1ccc(-c2nc(-c3ccccc3)nc(-c3cccc4c3C3(c5ccccc5-c5ccccc53)c3ccccc3-4)n2)cc1]

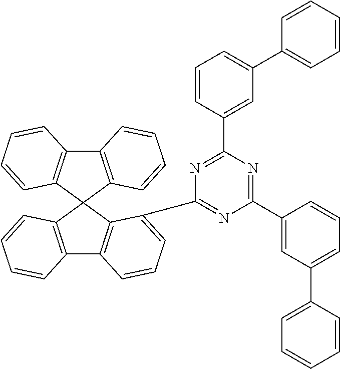 [SMILES: c1ccc(-c2cccc(-c3nc(-c4cccc(-c5ccccc5)c4)nc(-c4cccc5c4C4(c6ccccc6-c6ccccc64)c4ccccc4-5)n3)c2)cc1]

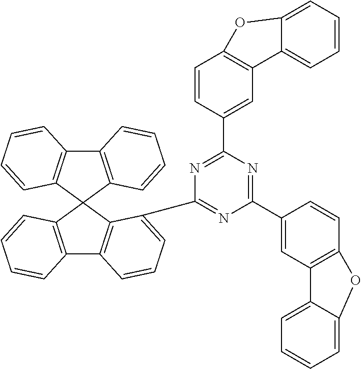 [SMILES: c1ccc2c(c1)-c1ccccc1C21c2ccccc2-c2cccc(-c3nc(-c4ccc5oc6ccccc6c5c4)nc(-c4ccc5oc6ccccc6c5c4)n3)c21]

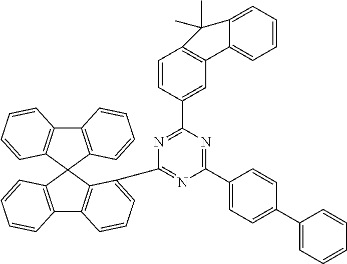 [SMILES: CC1(C)c2ccccc2-c2cc(-c3nc(-c4ccc(-c5ccccc5)cc4)nc(-c4cccc5c4C4(c6ccccc6-c6ccccc64)c4ccccc4-5)n3)ccc21]

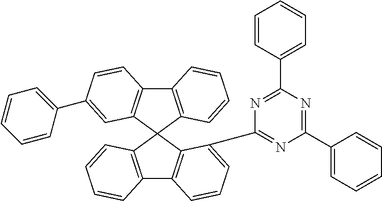 [SMILES: c1ccc(-c2ccc3c(c2)C2(c4ccccc4-3)c3ccccc3-c3cccc(-c4nc(-c5ccccc5)nc(-c5ccccc5)n4)c32)cc1]

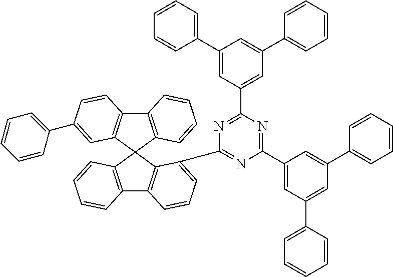 [SMILES: c1ccc(-c2cc(-c3ccccc3)cc(-c3nc(-c4cc(-c5ccccc5)cc(-c5ccccc5)c4)nc(-c4cccc5c4C4(c6ccccc6-c6ccc(-c7ccccc7)cc64)c4ccccc4-5)n3)c2)cc1]

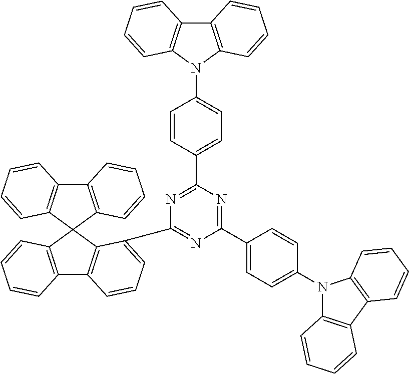 [SMILES: c1ccc2c(c1)-c1ccccc1C21c2ccccc2-c2cccc(-c3nc(-c4ccc(-n5c6ccccc6c6ccccc65)cc4)nc(-c4ccc(-n5c6ccccc6c6ccccc65)cc4)n3)c21]

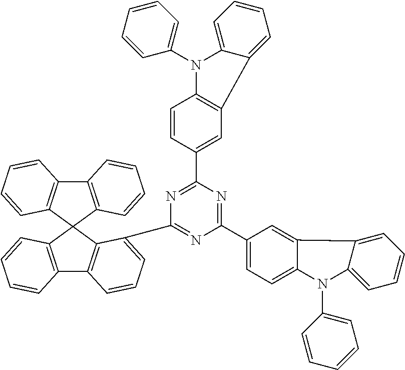 [SMILES: c1ccc(-n2c3ccccc3c3cc(-c4nc(-c5ccc6c(c5)c5ccccc5n6-c5ccccc5)nc(-c5cccc6c5C5(c7ccccc7-c7ccccc75)c5ccccc5-6)n4)ccc32)cc1]

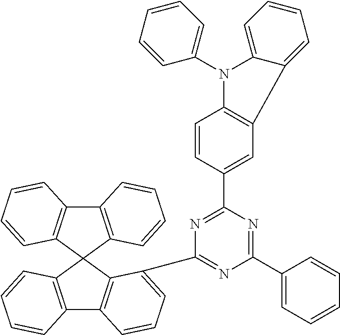 [SMILES: c1ccc(-c2nc(-c3ccc4c(c3)c3ccccc3n4-c3ccccc3)nc(-c3cccc4c3C3(c5ccccc5-c5ccccc53)c3ccccc3-4)n2)cc1]

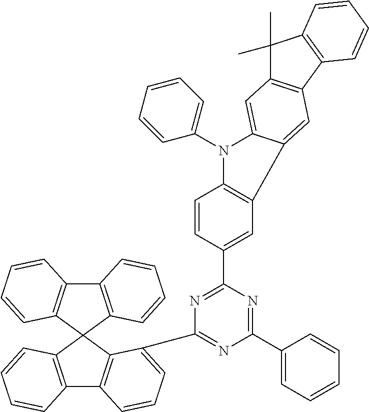 [SMILES: CC1(C)c2ccccc2-c2cc3c4cc(-c5nc(-c6ccccc6)nc(-c6cccc7c6C6(c8ccccc8-c8ccccc86)c6ccccc6-7)n5)ccc4n(-c4ccccc4)c3cc21]

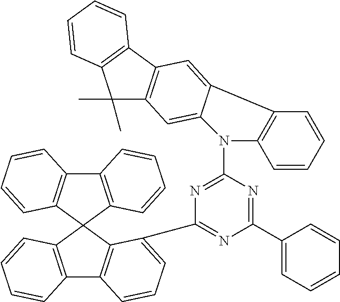 [SMILES: CC1(C)c2ccccc2-c2cc3c4ccccc4n(-c4nc(-c5ccccc5)nc(-c5cccc6c5C5(c7ccccc7-c7ccccc75)c5ccccc5-6)n4)c3cc21]

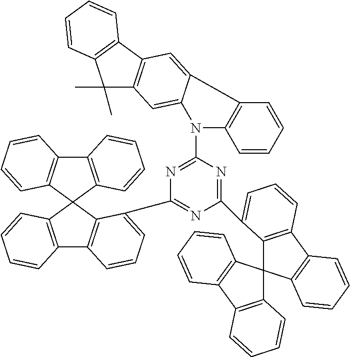 [SMILES: CC1(C)c2ccccc2-c2cc3c4ccccc4n(-c4nc(-c5cccc6c5C5(c7ccccc7-c7ccccc75)c5ccccc5-6)nc(-c5cccc6c5C5(c7ccccc7-c7ccccc75)c5ccccc5-6)n4)c3cc21]

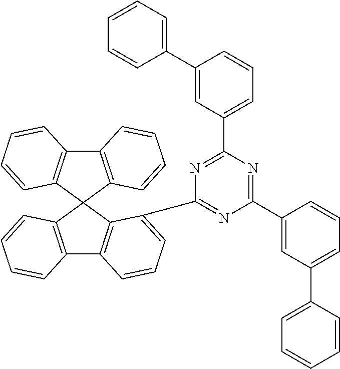 [SMILES: c1ccc(-c2cccc(-c3nc(-c4cccc(-c5ccccc5)c4)nc(-c4cccc5c4C4(c6ccccc6-c6ccccc64)c4ccccc4-5)n3)c2)cc1]

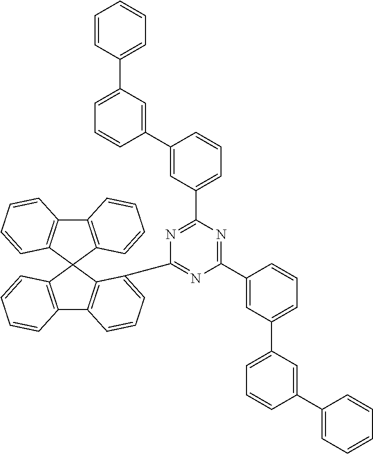 [SMILES: c1ccc(-c2cccc(-c3cccc(-c4nc(-c5cccc(-c6cccc(-c7ccccc7)c6)c5)nc(-c5cccc6c5C5(c7ccccc7-c7ccccc75)c5ccccc5-6)n4)c3)c2)cc1]

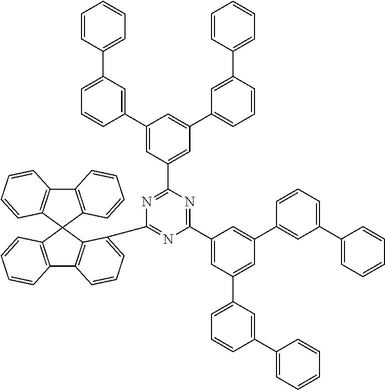 [SMILES: c1ccc(-c2cccc(-c3cc(-c4cccc(-c5ccccc5)c4)cc(-c4nc(-c5cc(-c6cccc(-c7ccccc7)c6)cc(-c6cccc(-c7ccccc7)c6)c5)nc(-c5cccc6c5C5(c7ccccc7-c7ccccc75)c5ccccc5-6)n4)c3)c2)cc1]

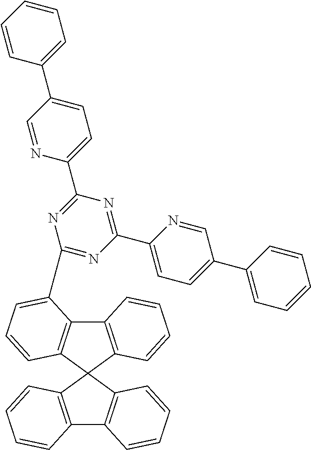 [SMILES: c1ccc(-c2ccc(-c3nc(-c4ccc(-c5ccccc5)cn4)nc(-c4cccc5c4-c4ccccc4C54c5ccccc5-c5ccccc54)n3)nc2)cc1]

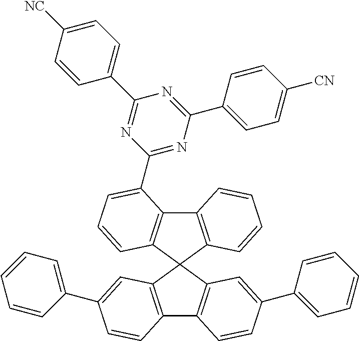 [SMILES: N#Cc1ccc(-c2nc(-c3ccc(C#N)cc3)nc(-c3cccc4c3-c3ccccc3C43c4cc(-c5ccccc5)ccc4-c4ccc(-c5ccccc5)cc43)n2)cc1]

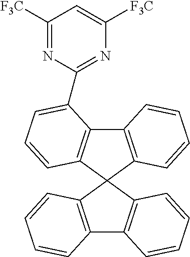 [SMILES: FC(F)(F)c1cc(C(F)(F)F)nc(-c2cccc3c2-c2ccccc2C32c3ccccc3-c3ccccc32)n1]

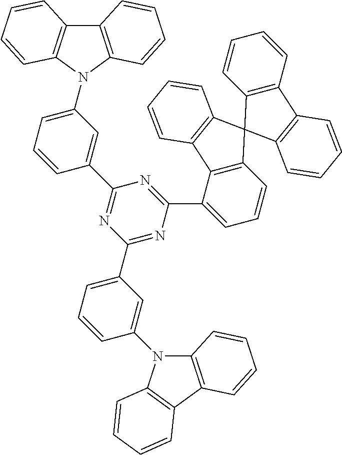 [SMILES: c1cc(-c2nc(-c3cccc(-n4c5ccccc5c5ccccc54)c3)nc(-c3cccc4c3-c3ccccc3C43c4ccccc4-c4ccccc43)n2)cc(-n2c3ccccc3c3ccccc32)c1]

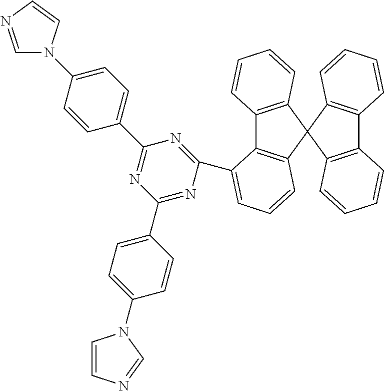 [SMILES: c1ccc2c(c1)-c1ccccc1C21c2ccccc2-c2c(-c3nc(-c4ccc(-n5ccnc5)cc4)nc(-c4ccc(-n5ccnc5)cc4)n3)cccc21]

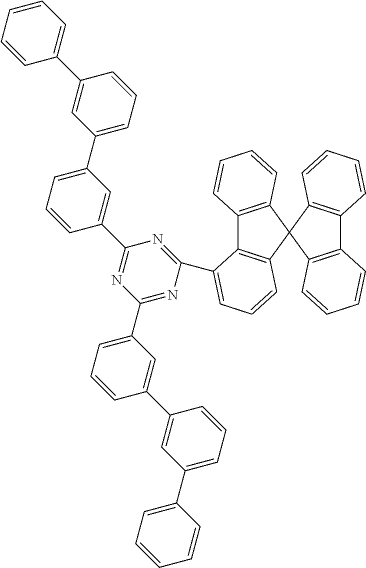 [SMILES: c1ccc(-c2cccc(-c3cccc(-c4nc(-c5cccc(-c6cccc(-c7ccccc7)c6)c5)nc(-c5cccc6c5-c5ccccc5C65c6ccccc6-c6ccccc65)n4)c3)c2)cc1]

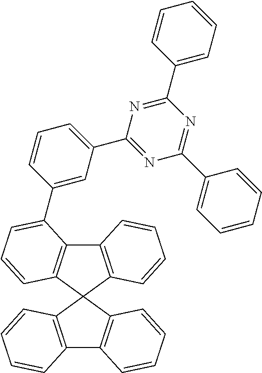 [SMILES: c1ccc(-c2nc(-c3ccccc3)nc(-c3cccc(-c4cccc5c4-c4ccccc4C54c5ccccc5-c5ccccc54)c3)n2)cc1]

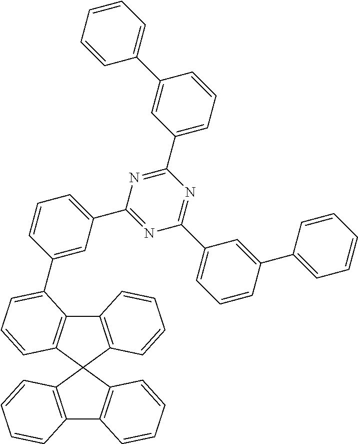 [SMILES: c1ccc(-c2cccc(-c3nc(-c4cccc(-c5ccccc5)c4)nc(-c4cccc(-c5cccc6c5-c5ccccc5C65c6ccccc6-c6ccccc65)c4)n3)c2)cc1]

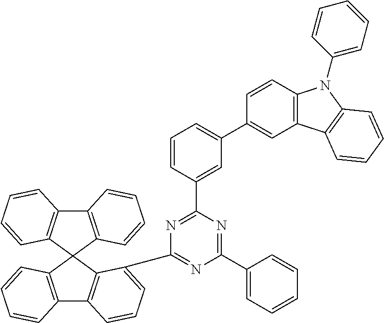 [SMILES: c1ccc(-c2nc(-c3cccc(-c4ccc5c(c4)c4ccccc4n5-c4ccccc4)c3)nc(-c3cccc4c3C3(c5ccccc5-c5ccccc53)c3ccccc3-4)n2)cc1]

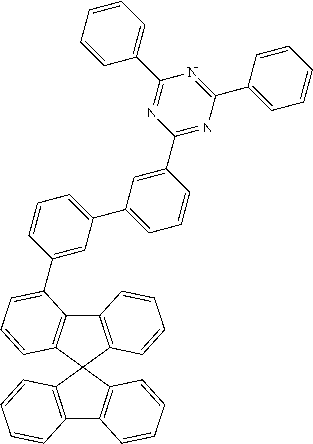 [SMILES: c1ccc(-c2nc(-c3ccccc3)nc(-c3cccc(-c4cccc(-c5cccc6c5-c5ccccc5C65c6ccccc6-c6ccccc65)c4)c3)n2)cc1]

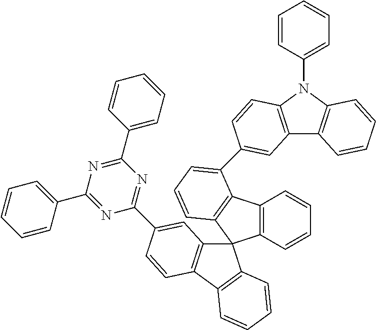 [SMILES: c1ccc(-c2nc(-c3ccccc3)nc(-c3ccc4c(c3)C3(c5ccccc5-4)c4ccccc4-c4c(-c5ccc6c(c5)c5ccccc5n6-c5ccccc5)cccc43)n2)cc1]

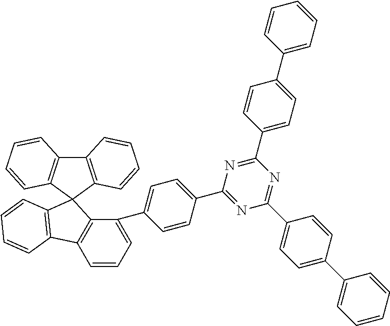 [SMILES: c1ccc(-c2ccc(-c3nc(-c4ccc(-c5ccccc5)cc4)nc(-c4ccc(-c5cccc6c5C5(c7ccccc7-c7ccccc75)c5ccccc5-6)cc4)n3)cc2)cc1]

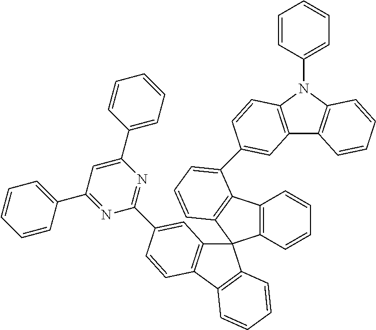 [SMILES: c1ccc(-c2cc(-c3ccccc3)nc(-c3ccc4c(c3)C3(c5ccccc5-4)c4ccccc4-c4c(-c5ccc6c(c5)c5ccccc5n6-c5ccccc5)cccc43)n2)cc1]

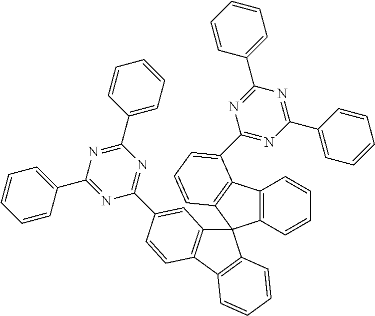 [SMILES: c1ccc(-c2nc(-c3ccccc3)nc(-c3ccc4c(c3)C3(c5ccccc5-4)c4ccccc4-c4c(-c5nc(-c6ccccc6)nc(-c6ccccc6)n5)cccc43)n2)cc1]

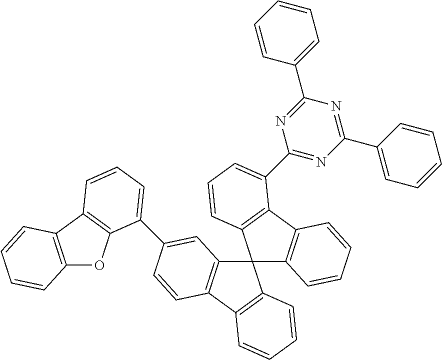 [SMILES: c1ccc(-c2nc(-c3ccccc3)nc(-c3cccc4c3-c3ccccc3C43c4ccccc4-c4ccc(-c5cccc6c5oc5ccccc56)cc43)n2)cc1]

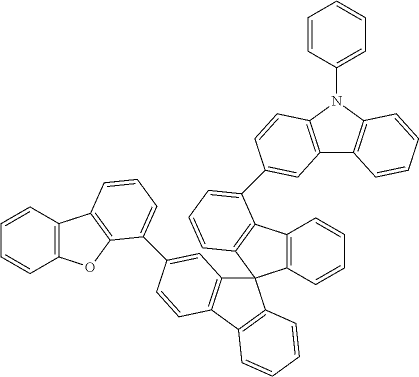 [SMILES: c1ccc(-n2c3ccccc3c3cc(-c4cccc5c4-c4ccccc4C54c5ccccc5-c5ccc(-c6cccc7c6oc6ccccc67)cc54)ccc32)cc1]

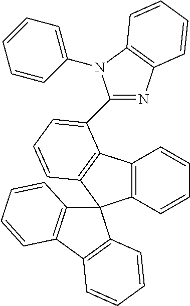 [SMILES: c1ccc(-n2c(-c3cccc4c3-c3ccccc3C43c4ccccc4-c4ccccc43)nc3ccccc32)cc1]

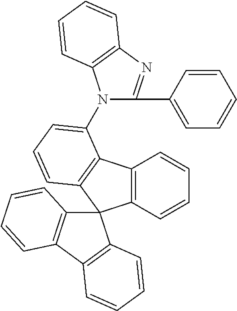 [SMILES: c1ccc(-c2nc3ccccc3n2-c2cccc3c2-c2ccccc2C32c3ccccc3-c3ccccc32)cc1]

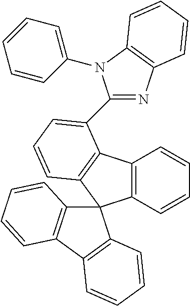 [SMILES: c1ccc(-n2c(-c3cccc4c3-c3ccccc3C43c4ccccc4-c4ccccc43)nc3ccccc32)cc1]

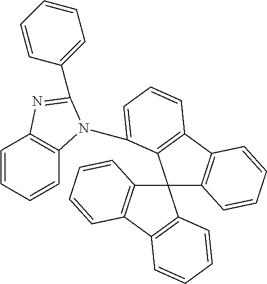 [SMILES: c1ccc(-c2nc3ccccc3n2-c2cccc3c2C2(c4ccccc4-c4ccccc42)c2ccccc2-3)cc1]

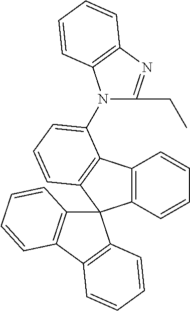 [SMILES: CCc1nc2ccccc2n1-c1cccc2c1-c1ccccc1C21c2ccccc2-c2ccccc21]

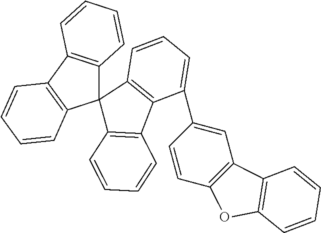 [SMILES: c1ccc2c(c1)-c1ccccc1C21c2ccccc2-c2c(-c3ccc4oc5ccccc5c4c3)cccc21]

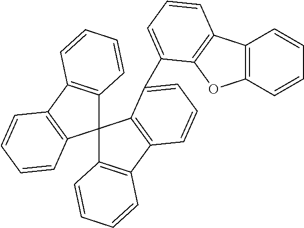 [SMILES: c1ccc2c(c1)-c1ccccc1C21c2ccccc2-c2cccc(-c3cccc4c3oc3ccccc34)c21]

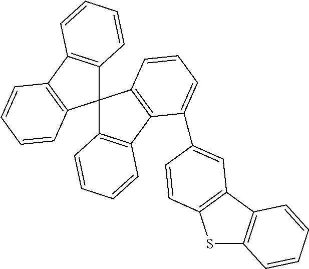 [SMILES: c1ccc2c(c1)-c1ccccc1C21c2ccccc2-c2c(-c3ccc4sc5ccccc5c4c3)cccc21]

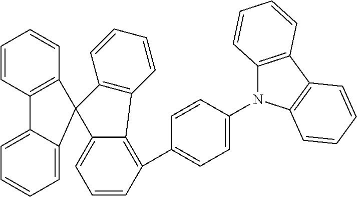 [SMILES: c1ccc2c(c1)-c1ccccc1C21c2ccccc2-c2c(-c3ccc(-n4c5ccccc5c5ccccc54)cc3)cccc21]

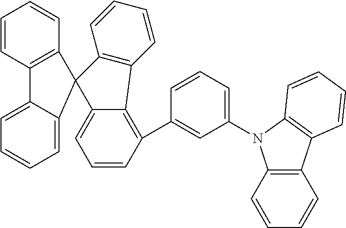 [SMILES: c1cc(-c2cccc3c2-c2ccccc2C32c3ccccc3-c3ccccc32)cc(-n2c3ccccc3c3ccccc32)c1]

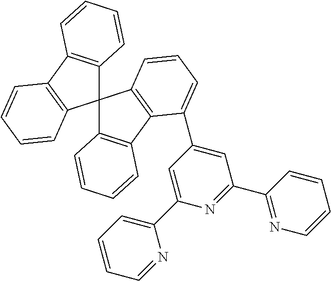 [SMILES: c1ccc(-c2cc(-c3cccc4c3-c3ccccc3C43c4ccccc4-c4ccccc43)cc(-c3ccccn3)n2)nc1]

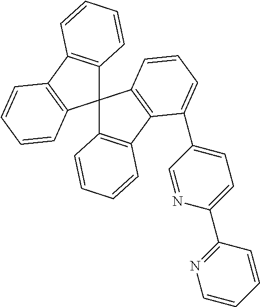 [SMILES: c1ccc(-c2ccc(-c3cccc4c3-c3ccccc3C43c4ccccc4-c4ccccc43)cn2)nc1]

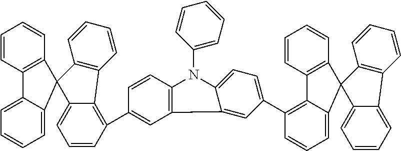 [SMILES: c1ccc(-n2c3ccc(-c4cccc5c4-c4ccccc4C54c5ccccc5-c5ccccc54)cc3c3cc(-c4cccc5c4-c4ccccc4C54c5ccccc5-c5ccccc54)ccc32)cc1]

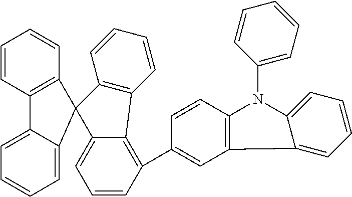 [SMILES: c1ccc(-n2c3ccccc3c3cc(-c4cccc5c4-c4ccccc4C54c5ccccc5-c5ccccc54)ccc32)cc1]

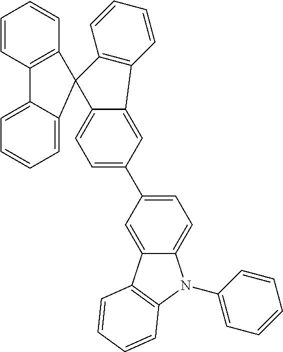 [SMILES: c1ccc(-n2c3ccccc3c3cc(-c4ccc5c(c4)-c4ccccc4C54c5ccccc5-c5ccccc54)ccc32)cc1]

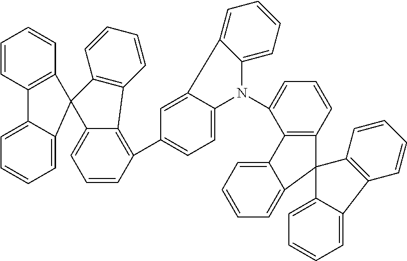 [SMILES: c1ccc2c(c1)-c1ccccc1C21c2ccccc2-c2c(-c3ccc4c(c3)c3ccccc3n4-c3cccc4c3-c3ccccc3C43c4ccccc4-c4ccccc43)cccc21]

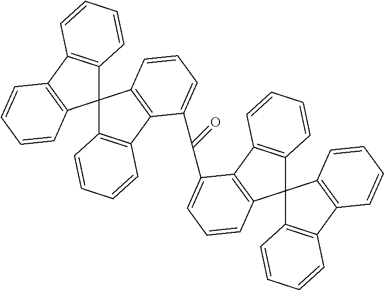 [SMILES: O=C(c1cccc2c1-c1ccccc1C21c2ccccc2-c2ccccc21)c1cccc2c1-c1ccccc1C21c2ccccc2-c2ccccc21]

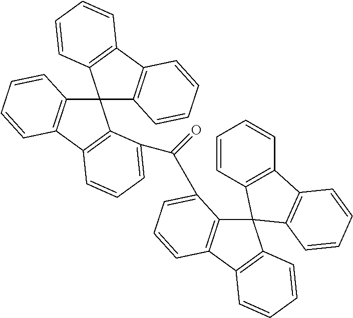 [SMILES: O=C(c1cccc2c1C1(c3ccccc3-c3ccccc31)c1ccccc1-2)c1cccc2c1C1(c3ccccc3-c3ccccc31)c1ccccc1-2]

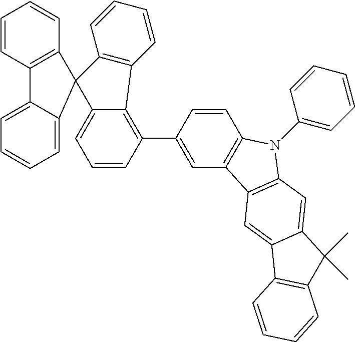 [SMILES: CC1(C)c2ccccc2-c2cc3c4cc(-c5cccc6c5-c5ccccc5C65c6ccccc6-c6ccccc65)ccc4n(-c4ccccc4)c3cc21]

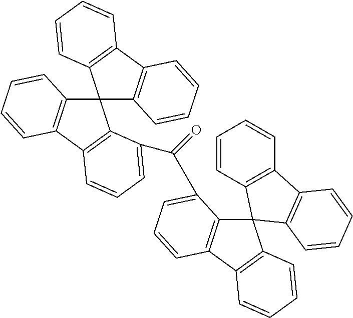 [SMILES: O=C(c1cccc2c1C1(c3ccccc3-c3ccccc31)c1ccccc1-2)c1cccc2c1C1(c3ccccc3-c3ccccc31)c1ccccc1-2]

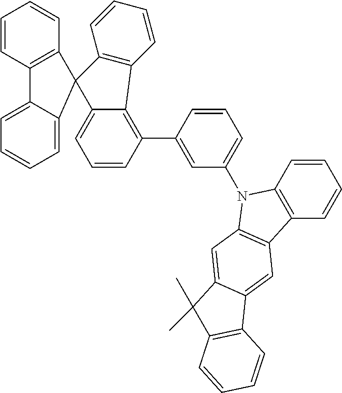 [SMILES: CC1(C)c2ccccc2-c2cc3c4ccccc4n(-c4cccc(-c5cccc6c5-c5ccccc5C65c6ccccc6-c6ccccc65)c4)c3cc21]

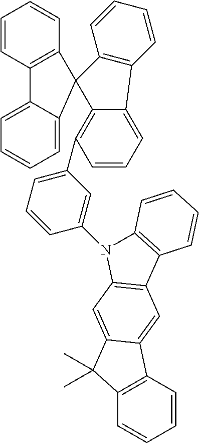 [SMILES: CC1(C)c2ccccc2-c2cc3c4ccccc4n(-c4cccc(-c5cccc6c5C5(c7ccccc7-c7ccccc75)c5ccccc5-6)c4)c3cc21]

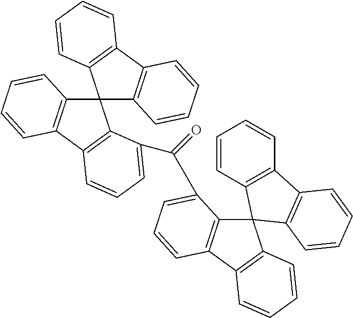 [SMILES: O=C(c1cccc2c1C1(c3ccccc3-c3ccccc31)c1ccccc1-2)c1cccc2c1C1(c3ccccc3-c3ccccc31)c1ccccc1-2]

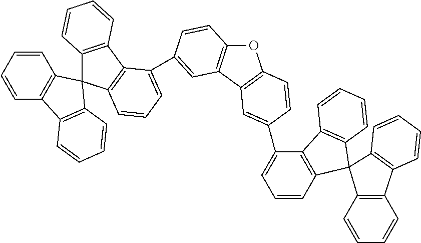 [SMILES: c1ccc2c(c1)-c1ccccc1C21c2ccccc2-c2c(-c3ccc4oc5ccc(-c6cccc7c6-c6ccccc6C76c7ccccc7-c7ccccc76)cc5c4c3)cccc21]

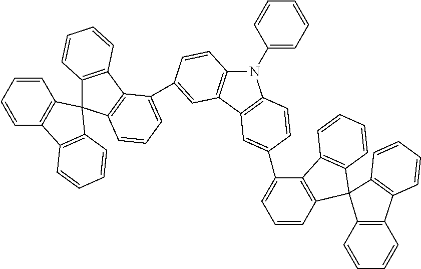 [SMILES: c1ccc(-n2c3ccc(-c4cccc5c4-c4ccccc4C54c5ccccc5-c5ccccc54)cc3c3cc(-c4cccc5c4-c4ccccc4C54c5ccccc5-c5ccccc54)ccc32)cc1]

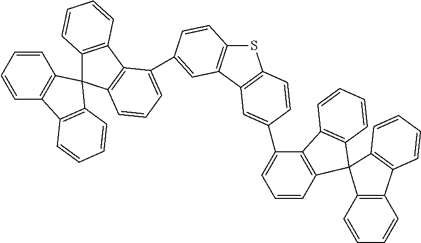 [SMILES: c1ccc2c(c1)-c1ccccc1C21c2ccccc2-c2c(-c3ccc4sc5ccc(-c6cccc7c6-c6ccccc6C76c7ccccc7-c7ccccc76)cc5c4c3)cccc21]

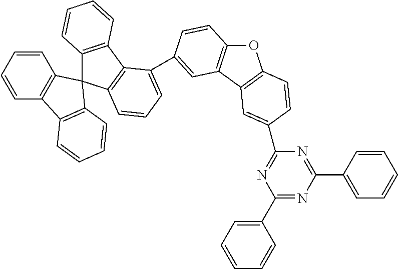 [SMILES: c1ccc(-c2nc(-c3ccccc3)nc(-c3ccc4oc5ccc(-c6cccc7c6-c6ccccc6C76c7ccccc7-c7ccccc76)cc5c4c3)n2)cc1]

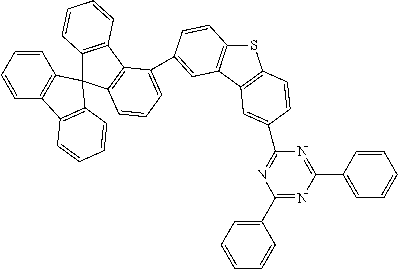 [SMILES: c1ccc(-c2nc(-c3ccccc3)nc(-c3ccc4sc5ccc(-c6cccc7c6-c6ccccc6C76c7ccccc7-c7ccccc76)cc5c4c3)n2)cc1]

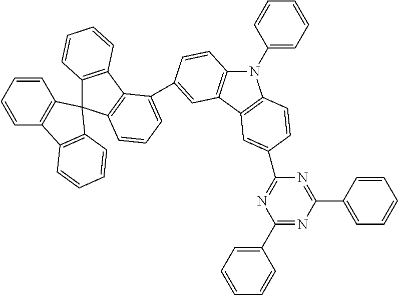 [SMILES: c1ccc(-c2nc(-c3ccccc3)nc(-c3ccc4c(c3)c3cc(-c5cccc6c5-c5ccccc5C65c6ccccc6-c6ccccc65)ccc3n4-c3ccccc3)n2)cc1]

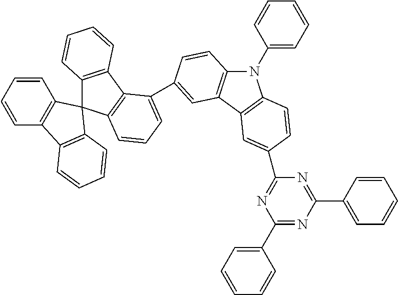 [SMILES: c1ccc(-c2nc(-c3ccccc3)nc(-c3ccc4c(c3)c3cc(-c5cccc6c5-c5ccccc5C65c6ccccc6-c6ccccc65)ccc3n4-c3ccccc3)n2)cc1]

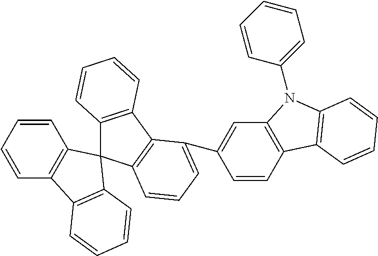 [SMILES: c1ccc(-n2c3ccccc3c3ccc(-c4cccc5c4-c4ccccc4C54c5ccccc5-c5ccccc54)cc32)cc1]

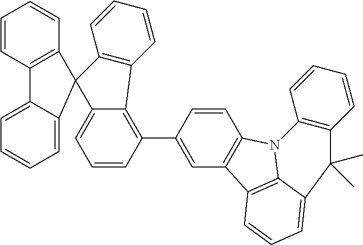 [SMILES: CC1(C)c2ccccc2-n2c3ccc(-c4cccc5c4-c4ccccc4C54c5ccccc5-c5ccccc54)cc3c3cccc1c32]

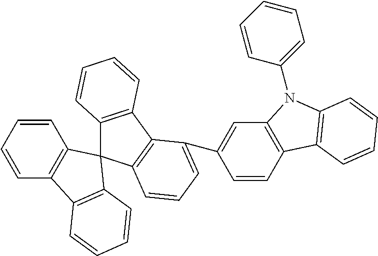 [SMILES: c1ccc(-n2c3ccccc3c3ccc(-c4cccc5c4-c4ccccc4C54c5ccccc5-c5ccccc54)cc32)cc1]

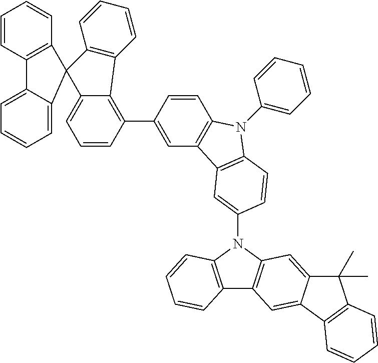 [SMILES: CC1(C)c2ccccc2-c2cc3c4ccccc4n(-c4ccc5c(c4)c4cc(-c6cccc7c6-c6ccccc6C76c7ccccc7-c7ccccc76)ccc4n5-c4ccccc4)c3cc21]

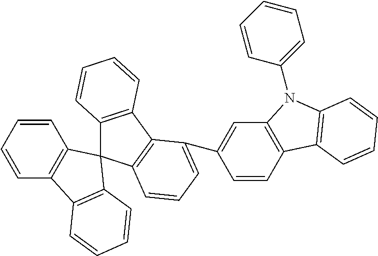 [SMILES: c1ccc(-n2c3ccccc3c3ccc(-c4cccc5c4-c4ccccc4C54c5ccccc5-c5ccccc54)cc32)cc1]

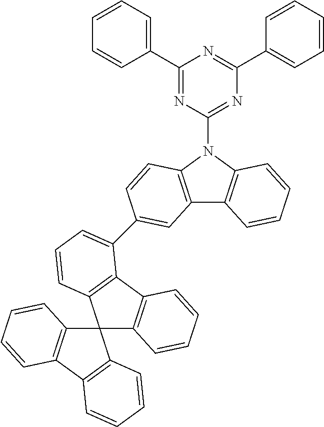 [SMILES: c1ccc(-c2nc(-c3ccccc3)nc(-n3c4ccccc4c4cc(-c5cccc6c5-c5ccccc5C65c6ccccc6-c6ccccc65)ccc43)n2)cc1]

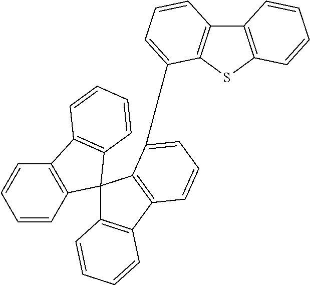 [SMILES: c1ccc2c(c1)-c1ccccc1C21c2ccccc2-c2cccc(-c3cccc4c3sc3ccccc34)c21]

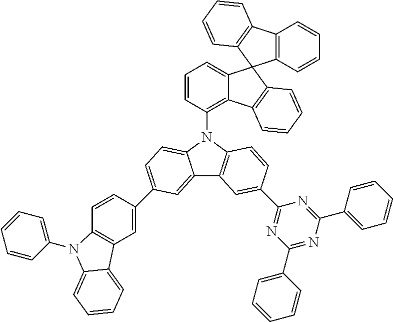 [SMILES: c1ccc(-c2nc(-c3ccccc3)nc(-c3ccc4c(c3)c3cc(-c5ccc6c(c5)c5ccccc5n6-c5ccccc5)ccc3n4-c3cccc4c3-c3ccccc3C43c4ccccc4-c4ccccc43)n2)cc1]

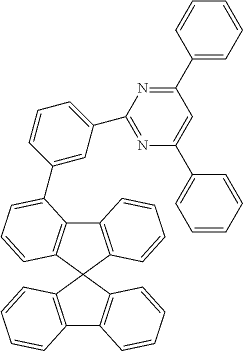 [SMILES: c1ccc(-c2cc(-c3ccccc3)nc(-c3cccc(-c4cccc5c4-c4ccccc4C54c5ccccc5-c5ccccc54)c3)n2)cc1]

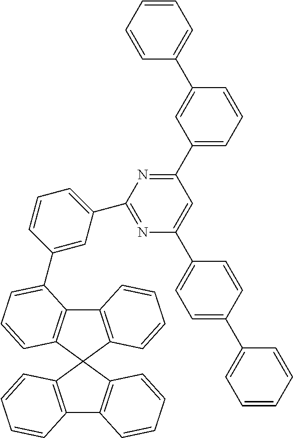 [SMILES: c1ccc(-c2ccc(-c3cc(-c4cccc(-c5ccccc5)c4)nc(-c4cccc(-c5cccc6c5-c5ccccc5C65c6ccccc6-c6ccccc65)c4)n3)cc2)cc1]

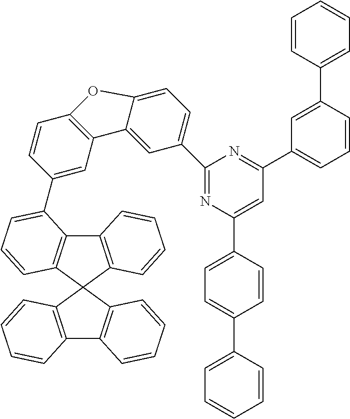 [SMILES: c1ccc(-c2ccc(-c3cc(-c4cccc(-c5ccccc5)c4)nc(-c4ccc5oc6ccc(-c7cccc8c7-c7ccccc7C87c8ccccc8-c8ccccc87)cc6c5c4)n3)cc2)cc1]

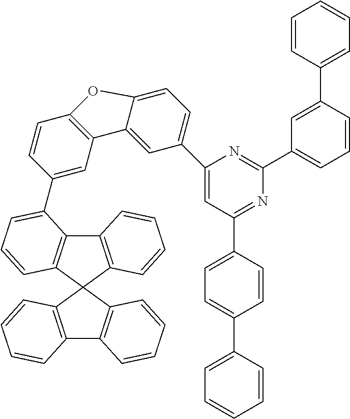 [SMILES: c1ccc(-c2ccc(-c3cc(-c4ccc5oc6ccc(-c7cccc8c7-c7ccccc7C87c8ccccc8-c8ccccc87)cc6c5c4)nc(-c4cccc(-c5ccccc5)c4)n3)cc2)cc1]

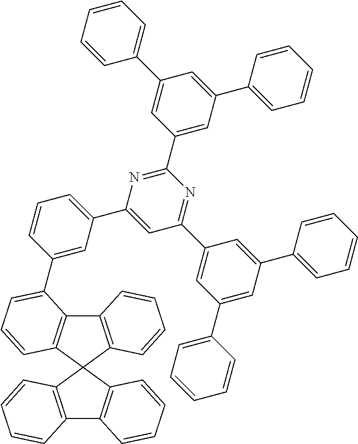 [SMILES: c1ccc(-c2cc(-c3ccccc3)cc(-c3cc(-c4cccc(-c5cccc6c5-c5ccccc5C65c6ccccc6-c6ccccc65)c4)nc(-c4cc(-c5ccccc5)cc(-c5ccccc5)c4)n3)c2)cc1]

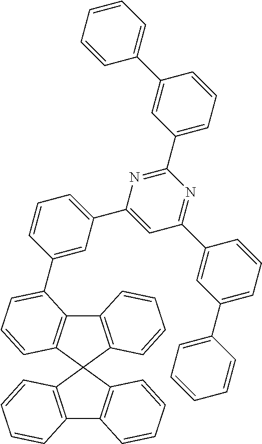 [SMILES: c1ccc(-c2cccc(-c3cc(-c4cccc(-c5cccc6c5-c5ccccc5C65c6ccccc6-c6ccccc65)c4)nc(-c4cccc(-c5ccccc5)c4)n3)c2)cc1]

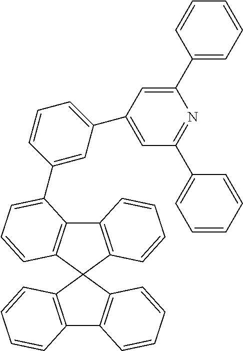 [SMILES: c1ccc(-c2cc(-c3cccc(-c4cccc5c4-c4ccccc4C54c5ccccc5-c5ccccc54)c3)cc(-c3ccccc3)n2)cc1]

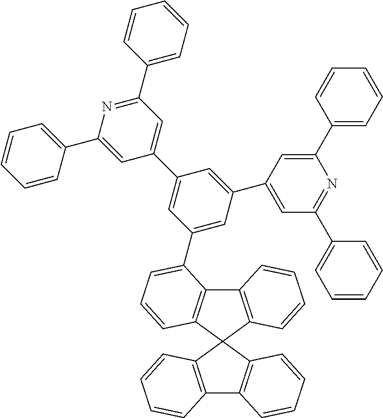 [SMILES: c1ccc(-c2cc(-c3cc(-c4cc(-c5ccccc5)nc(-c5ccccc5)c4)cc(-c4cccc5c4-c4ccccc4C54c5ccccc5-c5ccccc54)c3)cc(-c3ccccc3)n2)cc1]

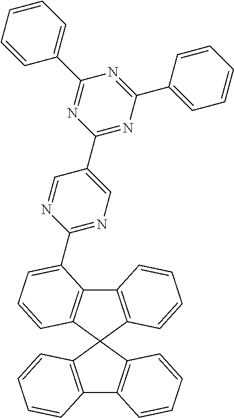 [SMILES: c1ccc(-c2nc(-c3ccccc3)nc(-c3cnc(-c4cccc5c4-c4ccccc4C54c5ccccc5-c5ccccc54)nc3)n2)cc1]

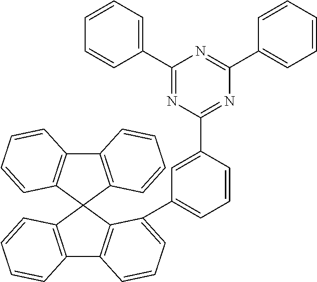 [SMILES: c1ccc(-c2nc(-c3ccccc3)nc(-c3cccc(-c4cccc5c4C4(c6ccccc6-c6ccccc64)c4ccccc4-5)c3)n2)cc1]

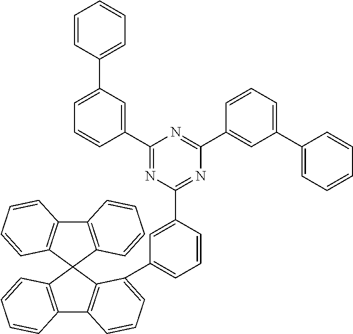 [SMILES: c1ccc(-c2cccc(-c3nc(-c4cccc(-c5ccccc5)c4)nc(-c4cccc(-c5cccc6c5C5(c7ccccc7-c7ccccc75)c5ccccc5-6)c4)n3)c2)cc1]

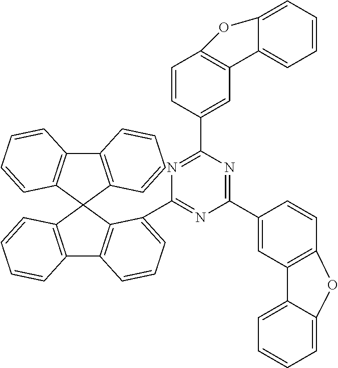 [SMILES: c1ccc2c(c1)-c1ccccc1C21c2ccccc2-c2cccc(-c3nc(-c4ccc5oc6ccccc6c5c4)nc(-c4ccc5oc6ccccc6c5c4)n3)c21]

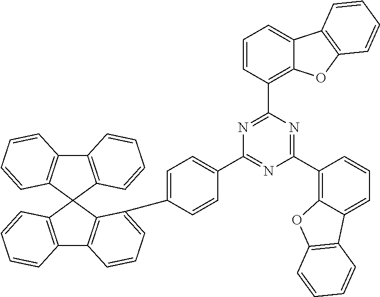 [SMILES: c1ccc2c(c1)-c1ccccc1C21c2ccccc2-c2cccc(-c3ccc(-c4nc(-c5cccc6c5oc5ccccc56)nc(-c5cccc6c5oc5ccccc56)n4)cc3)c21]

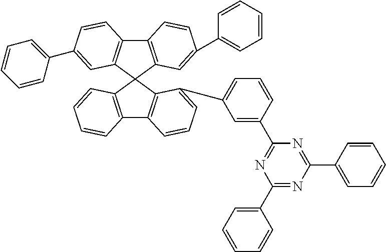 [SMILES: c1ccc(-c2ccc3c(c2)C2(c4cc(-c5ccccc5)ccc4-3)c3ccccc3-c3cccc(-c4cccc(-c5nc(-c6ccccc6)nc(-c6ccccc6)n5)c4)c32)cc1]

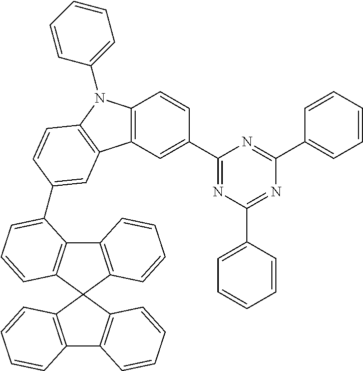 [SMILES: c1ccc(-c2nc(-c3ccccc3)nc(-c3ccc4c(c3)c3cc(-c5cccc6c5-c5ccccc5C65c6ccccc6-c6ccccc65)ccc3n4-c3ccccc3)n2)cc1]

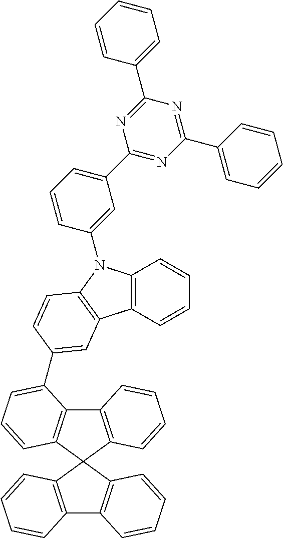 [SMILES: c1ccc(-c2nc(-c3ccccc3)nc(-c3cccc(-n4c5ccccc5c5cc(-c6cccc7c6-c6ccccc6C76c7ccccc7-c7ccccc76)ccc54)c3)n2)cc1]

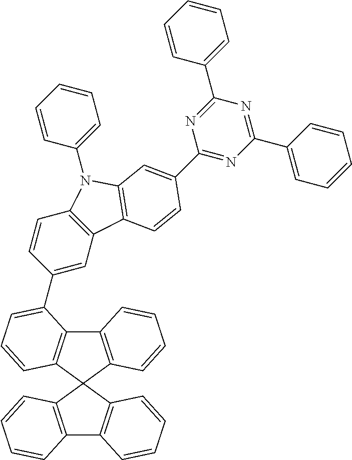 [SMILES: c1ccc(-c2nc(-c3ccccc3)nc(-c3ccc4c5cc(-c6cccc7c6-c6ccccc6C76c7ccccc7-c7ccccc76)ccc5n(-c5ccccc5)c4c3)n2)cc1]

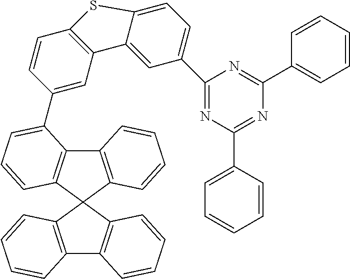 [SMILES: c1ccc(-c2nc(-c3ccccc3)nc(-c3ccc4sc5ccc(-c6cccc7c6-c6ccccc6C76c7ccccc7-c7ccccc76)cc5c4c3)n2)cc1]

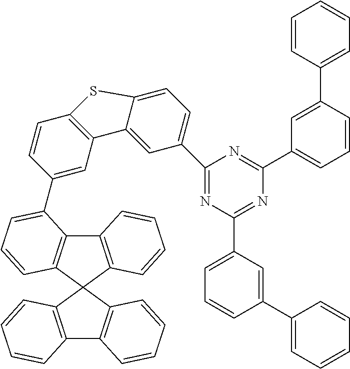 [SMILES: c1ccc(-c2cccc(-c3nc(-c4cccc(-c5ccccc5)c4)nc(-c4ccc5sc6ccc(-c7cccc8c7-c7ccccc7C87c8ccccc8-c8ccccc87)cc6c5c4)n3)c2)cc1]

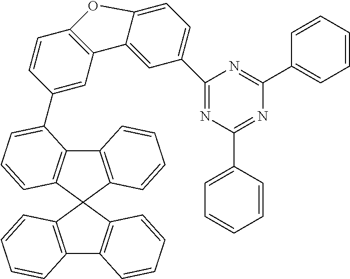 [SMILES: c1ccc(-c2nc(-c3ccccc3)nc(-c3ccc4oc5ccc(-c6cccc7c6-c6ccccc6C76c7ccccc7-c7ccccc76)cc5c4c3)n2)cc1]

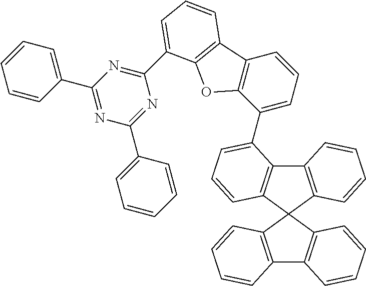 [SMILES: c1ccc(-c2nc(-c3ccccc3)nc(-c3cccc4c3oc3c(-c5cccc6c5-c5ccccc5C65c6ccccc6-c6ccccc65)cccc34)n2)cc1]

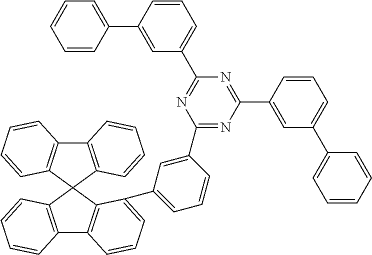 [SMILES: c1ccc(-c2cccc(-c3nc(-c4cccc(-c5ccccc5)c4)nc(-c4cccc(-c5cccc6c5C5(c7ccccc7-c7ccccc75)c5ccccc5-6)c4)n3)c2)cc1]

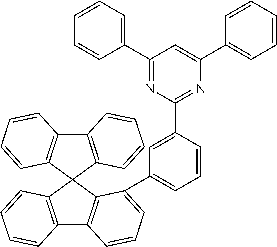 [SMILES: c1ccc(-c2cc(-c3ccccc3)nc(-c3cccc(-c4cccc5c4C4(c6ccccc6-c6ccccc64)c4ccccc4-5)c3)n2)cc1]

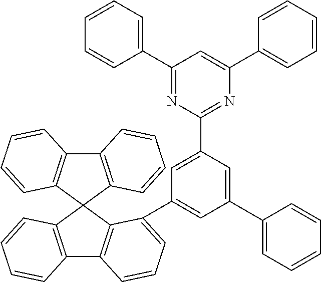 [SMILES: c1ccc(-c2cc(-c3nc(-c4ccccc4)cc(-c4ccccc4)n3)cc(-c3cccc4c3C3(c5ccccc5-c5ccccc53)c3ccccc3-4)c2)cc1]

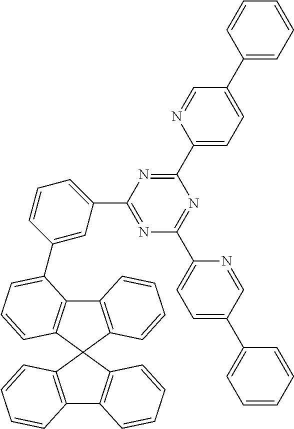 [SMILES: c1ccc(-c2ccc(-c3nc(-c4cccc(-c5cccc6c5-c5ccccc5C65c6ccccc6-c6ccccc65)c4)nc(-c4ccc(-c5ccccc5)cn4)n3)nc2)cc1]

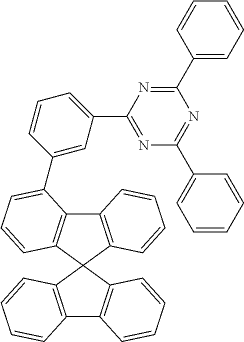 [SMILES: c1ccc(-c2nc(-c3ccccc3)nc(-c3cccc(-c4cccc5c4-c4ccccc4C54c5ccccc5-c5ccccc54)c3)n2)cc1]

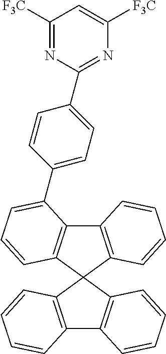 [SMILES: FC(F)(F)c1cc(C(F)(F)F)nc(-c2ccc(-c3cccc4c3-c3ccccc3C43c4ccccc4-c4ccccc43)cc2)n1]

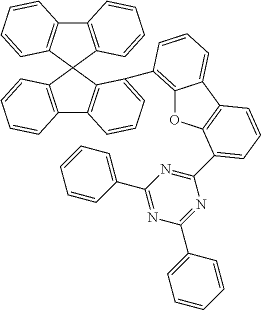 [SMILES: c1ccc(-c2nc(-c3ccccc3)nc(-c3cccc4c3oc3c(-c5cccc6c5C5(c7ccccc7-c7ccccc75)c5ccccc5-6)cccc34)n2)cc1]

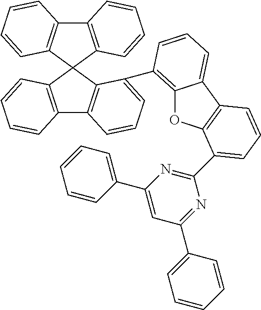 [SMILES: c1ccc(-c2cc(-c3ccccc3)nc(-c3cccc4c3oc3c(-c5cccc6c5C5(c7ccccc7-c7ccccc75)c5ccccc5-6)cccc34)n2)cc1]

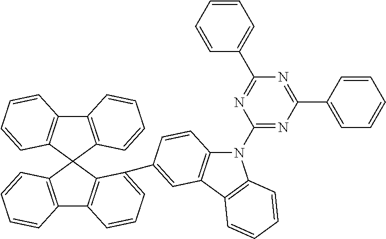 [SMILES: c1ccc(-c2nc(-c3ccccc3)nc(-n3c4ccccc4c4cc(-c5cccc6c5C5(c7ccccc7-c7ccccc75)c5ccccc5-6)ccc43)n2)cc1]

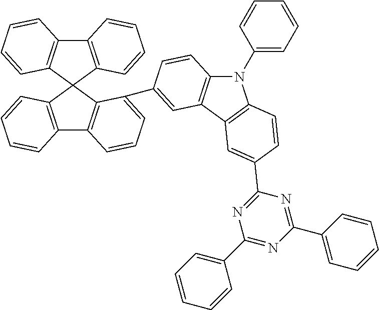 [SMILES: c1ccc(-c2nc(-c3ccccc3)nc(-c3ccc4c(c3)c3cc(-c5cccc6c5C5(c7ccccc7-c7ccccc75)c5ccccc5-6)ccc3n4-c3ccccc3)n2)cc1]

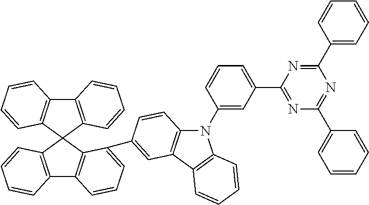 [SMILES: c1ccc(-c2nc(-c3ccccc3)nc(-c3cccc(-n4c5ccccc5c5cc(-c6cccc7c6C6(c8ccccc8-c8ccccc86)c6ccccc6-7)ccc54)c3)n2)cc1]

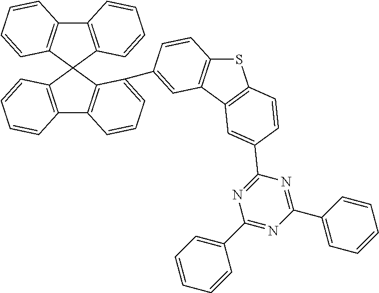 [SMILES: c1ccc(-c2nc(-c3ccccc3)nc(-c3ccc4sc5ccc(-c6cccc7c6C6(c8ccccc8-c8ccccc86)c6ccccc6-7)cc5c4c3)n2)cc1]

The compounds according to the invention can be prepared by synthesis steps known to the person skilled in the art, such as, for example, bromination, Ullmann arylation, Hartwig-Buchwald coupling, etc.

The syntheses here generally start from the 1- or 4-halogenated, in particular brominated spirobifluorene derivatives, followed by introduction of the group —Ar or -L-Ar, in particular by a metal-catalysed coupling reaction, for example a Suzuki coupling.

Another suitable leaving group, for example tosylate or triflate, can be used analogously instead of the halogen. The synthesis of spirobifluorene which is substituted by Ar in the 1-position is shown in Scheme 1. The synthesis of spirobifluorene which is substituted by Ar in the 4-position is shown in Scheme 2.

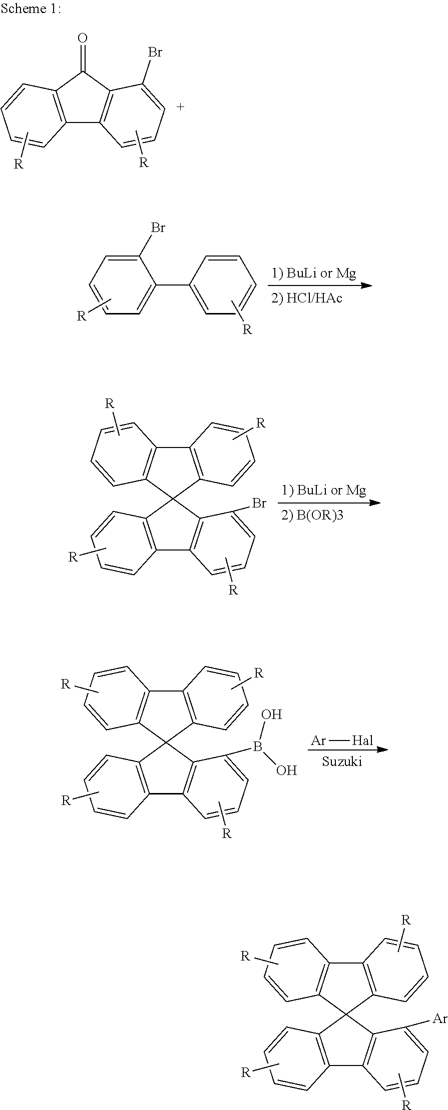

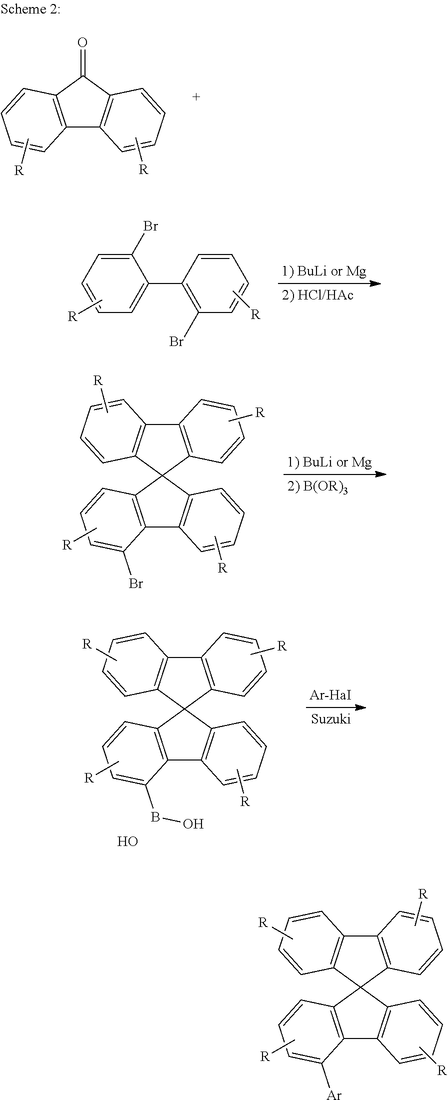

Corresponding compounds in which the group Ar is not bonded directly to the spirobifluorene, but instead via a group L which does not stand for a single bond can likewise also be synthesised entirely analogously by employing a corresponding compound Ar-L-Hal instead of a halogenated aromatic compound Ar-Hal. Hal here preferably stands for Cl, Br or I, in particular for Br.

The halogenated spirobifluorene derivatives which are coupled to a boronic acid derivative of the group -L-Ar can likewise be employed entirely analogously.

The present invention therefore furthermore relates to a process for the preparation of a compound of the formula (1) or (2), characterised in that the group -L-Ar is introduced by a metal-catalysed coupling reaction between a 1- or 4-functionalised spirobifluorene and a functionalised group -L-Ar. In a preferred embodiment of the invention, the spirobifluorene derivative is a halogen-functionalised compound and the group -L-Ar is a compound which is functionalised by means of a boronic acid derivative. In a further preferred embodiment of the invention, the spirobifluorene derivative is a compound which is functionalised by means of a boronic acid derivative, and the group -L-Ar is a halogen-functionalised compound.

The compounds according to the invention are suitable for use in an electronic device. An electronic device here is taken to mean a device which comprises at least one layer which comprises at least one organic compound. However, the component here may also comprise inorganic materials or also layers built up entirely from inorganic materials.

The present invention therefore furthermore relates to the use of the compounds according to the invention in an electronic device, in particular in an organic electroluminescent device.

The present invention still furthermore relates to an electronic device cornprising at least one compound according to the invention. The preferences stated above likewise apply to the electronic devices.

The electronic device is preferably selected from the group consisting of organic electroluminescent devices (organic light-emitting diodes, OLEDs), organic integrated circuits (O-ICs), organic field-effect transistors (O-FETs), organic thin-film transistors (O-TFTs), organic light-emitting transistors (O-LETs), organic solar cells (O-SCs), organic dye-sensitised solar cells (ODSSCs), organic optical detectors, organic photoreceptors, organic field-quench devices (O-FQDs), light-emitting electrochemical cells (LECs), organic laser diodes (O-lasers) and organic plasmon emitting devices (D. M. Koller et al., Nature Photonics 2008, 1-4), but preferably organic electroluminescent devices (OLEDs), particularly preferably phosphorescent OLEDs.

The organic electroluminescent devices and the light-emitting electrochemical cells can be employed for various applications, for example for monochromatic or polychromatic displays, for lighting applications or for medical and/or cosmetic applications, for example in phototherapy.

The organic electroluminescent device comprises a cathode, an anode and at least one emitting layer. Apart from these layers, it may also comprise further layers, for example in each case one or more hole-injection layers, hole-transport layers, hole-blocking layers, electron-transport layers, electron-injection layers, exciton-blocking layers, electron-blocking layers and/or charge-generation layers. Interlayers, which have, for example, an exciton-blocking function, may likewise be introduced between two emitting layers. However, it should be pointed out that each of these layers does not necessarily have to be present.

The organic electroluminescent device here may comprise one emitting layer or a plurality of emitting layers. If a plurality of emission layers is present, these preferably have in total a plurality of emission maxima between 380 nm and 750 nm, resulting overall in white emission, i.e. various emitting compounds which are able to fluoresce or phosphoresce are used in the emitting layers. Particular preference is given to systems having three emitting layers, where the three layers exhibit blue, green and orange or red emission (for the basic structure see, for example, WO 2005/011013). It is possible here for all emitting layers to be fluorescent or for all emitting layers to be phosphorescent or for one or more emitting layers to be fluorescent and one or more other layers to be phosphorescent.

The compound according to the invention in accordance with the embodiments indicated above can be employed here in different layers, depending on the precise structure. Preference is given to an organic electroluminescent device comprising a compound of the formula (1) or formula (2) or the preferred embodiments as electron-transport material in an electron-transport or hole-blocking layer or as matrix material for fluorescent or phosphorescent emitters, in particular for phosphorescent emitters. The preferred embodiments indicated above also apply to the use of the materials in organic electronic devices.

In a preferred embodiment of the invention, the compound of the formula (1) or formula (2) or the preferred embodiments is employed as electron-transport material in an electron-transport layer. The emitting layer here can be fluorescent or phosphorescent. Furthermore, the electron-transport layer may be directly adjacent to the anode, or an additional electron-injection layer may be present which is located between the cathode and the electron-transport layer. It is likewise possible for a plurality of electron-transport layers to be present, at least one layer of which comprises at least one compound of the formula (1) or (2).

In a further preferred embodiment of the invention, the compound of the formula (1) or formula (2) or the preferred embodiments is employed in a hole-blocking layer. A hole-blocking layer is taken to mean a layer which is directly adjacent to an emitting layer on the cathode side.

In a further preferred embodiment of the invention, the compound of the formula (1) or formula (2) or the preferred embodiments is employed as matrix material (=host material) for a fluorescent or phosphorescent compound, in particular for a phosphorescent compound, in an emitting layer. The organic electroluminescent device here may comprise one emitting layer or a plurality of emitting layers, where at least one emitting layer comprises at least one compound according to the invention as matrix material.

If the compound of the formula (1) or formula (2) or the preferred embodiments is employed as matrix material for an emitting compound in an emitting layer, it is preferably employed in combination with one or more phosphorescent materials (triplet emitters). Phosphorescence in the sense of this invention is taken to mean the luminescence from an excited state having a spin multiplicity >1, in particular from an excited triplet state. For the purposes of this application, all luminescent complexes containing transition metals or lanthanoids, in particular all luminescent iridium, platinum and copper complexes, are to be regarded as phosphorescent compounds.

The mixture comprising the compound of the formula (1) or formula (2) or the preferred embodiments and the emitting compound comprises between 99.9 and 1% by weight, preferably between 99 and 10% by weight, particularly preferably between 97 and 60% by weight, in particular between 95 and 80% by weight, of the compound of the formula (1) or formula (2) or the preferred embodiments, based on the entire mixture comprising emitter and matrix material. Correspondingly, the mixture comprises between 0.1 and 99% by weight, preferably between 1 and 90% by weight, particularly preferably between 3 and 40% by weight, in particular between 5 and 20% by weight, of the emitter, based on the entire mixture comprising emitter and matrix material. The limits indicated above apply, in particular, if the layer is applied from solution. If the layer is applied by vacuum evaporation, the same numerical values apply, with the percentage in this case being indicated in % by vol. in each case.

A particularly preferred embodiment of the present invention is the use of the compound of the formula (1) or formula (2) or the preferred embodiments as matrix material for a phosphorescent emitter in combination with a further matrix material. Particularly suitable matrix materials which can be employed in combination with the compounds of the formula (1) or formula (2) or the preferred embodiments are aromatic ketones, aromatic phosphine oxides or aromatic sulfoxides or sulfones, for example in accordance with WO 2004/013080, WO 2004/093207, WO 2006/005627 or WO 2010/006680, triarylamines, carbazole derivatives, for example CBP (N,N-bis-carbazolylbiphenyl), m-CBP or the carbazole derivatives disclosed in WO 2005/039246, US 2005/0069729, JP 2004/288381, EP 1205527 or WO 2008/086851, indolocarbazole derivatives, for example in accordance with WO 2007/063754 or WO 2008/056746, indenocarbazole derivatives, for example in accordance with WO 2010/136109 or WO 2011/000455, azacarbazole derivatives, for example in accordance with EP 1617710, EP 1617711, EP 1731584, JP 2005/347160, bipolar matrix materials, for example in accordance with WO 2007/137725, silanes, for example in accordance with WO 2005/111172, azaboroles or boronic esters, for example in accordance with WO 2006/117052, triazine derivatives, for example in accordance with WO 2010/015306, WO 2007/063754 or WO 08/056746, zinc complexes, for example in accordance with EP 652273 or WO 2009/062578, fluorene derivatives, for example in accordance with WO 2009/124627, diazasilole or tetraazasilole derivatives, for example in accordance with WO 2010/054729, diazaphosphole derivatives, for example in accordance with WO 2010/054730, or bridged carbazole derivatives, for example in accordance with US 2009/0136779, WO 2010/050778, WO 2011/042107 or WO 2011/088877. It is furthermore possible to use an electronically neutral co-host which has neither hole-transporting nor electron-transporting properties, as described, for example, in WO 2010/108579.

It is likewise possible to use two or more phosphorescent emitters in the mixture. In this case, the emitter which emits at shorter wavelength acts as co-host in the mixture.

Suitable phosphorescent compounds (=triplet emitters) are, in particular, compounds which emit light, preferably in the visible region, on suitable excitation and in addition contain at least one atom having an atomic number greater than 20, preferably greater than 38 and less than 84, particularly preferably greater than 56 and less than 80, in particular a metal having this atomic number. The phosphorescent emitters used are preferably compounds which contain copper, molybdenum, tungsten, rhenium, ruthenium, osmium, rhodium, iridium, palladium, platinum, silver, gold or europium, in particular compounds which contain iridium, platinum or copper.

Examples of the emitters described above are revealed by the applications WO 2000/70655, WO 2001/41512, WO 2002/02714, WO 2002/15645, EP 1191613, EP 1191612, EP 1191614, WO 2005/033244, WO 2005/019373, US 2005/0258742, WO 2009/146770, WO 2010/015307, WO 2010/031485, WO 2010/054731, WO 2010/054728, WO 2010/086089, WO 2010/099852, WO 2010/102709, WO 2011/157339 or WO 2012/007086. In general, all phosphorescent complexes as used in accordance with the prior art for phosphorescent OLEDs and as are known to the person skilled in the art in the area of organic electroluminescence are suitable, and the person skilled in the art will be able to use further phosphorescent complexes without inventive step.

In a further embodiment of the invention, the organic electroluminescent device according to the invention does not comprise a separate hole-injection layer and/or hole-transport layer and/or hole-blocking layer and/or electron-transport layer, i.e. the emitting layer is directly adjacent to the hole-injection layer or the anode, and/or the emitting layer is directly adjacent to the electron-transport layer or the electron-injection layer or the cathode, as described, for example, in WO 2005/053051. It is furthermore possible to use a metal complex which is identical or similar to the metal complex in the emitting layer as hole-transport or hole-injection material directly adjacent to the emitting layer, as described, for example, in WO 2009/030981.

It is furthermore possible to use the compound of the formula (1) or formula (2) or the preferred embodiments both in a hole-blocking layer or electron-transport layer and also as matrix in an emitting layer.

In the further layers of the organic electroluminescent device according to the invention, it is possible to use all materials as usually employed in accordance with the prior art. The person skilled in the art will therefore be able, without inventive step, to employ all materials known for organic electroluminescent devices in combination with the compounds of the formula (1) or formula (2) according to the invention or the preferred embodiments.

Preference is furthermore given to an organic electroluminescent device, characterised in that one or more layers are coated by means of a sublimation process, in which the materials are vapour-deposited in vacuum sublimation units at an initial pressure of usually less than 10⁻⁵ mbar, preferably less than 10⁻⁶ mbar. However, it is also possible for the initial pressure to be even lower, for example less than 10⁻⁷ mbar.

Preference is likewise given to an organic electroluminescent device, characterised in that one or more layers are coated by means of the OVPD (organic vapour phase deposition) process or with the aid of carrier-gas sublimation, in which the materials are applied at a pressure between 10⁻⁵ mbar and 1 bar. A special case of this process is the OVJP (organic vapour jet printing) process, in which the materials are applied directly through a nozzle and thus structured (for example M. S. Arnold et al., Appl. Phys. Lett. 2008, 92, 053301).

Preference is furthermore given to an organic electroluminescent device, characterised in that one or more layers are produced from solution, such as, for example, by spin coating, or by means of any desired printing process, such as, for example, LITI (light induced thermal imaging, thermal transfer printing), ink-jet printing, screen printing, flexographic printing, offset printing or nozzle printing. Soluble compounds, which are obtained, for example, by suitable substitution, are necessary for this purpose. These processes are also particularly suitable for the compounds according to the invention, since these generally have very good solubility in organic solvents.

Also possible are hybrid processes, in which, for example, one or more layers are applied from solution and one or more further layers are applied by vapour deposition. Thus, for example, the emitting layer can be applied from solution and the electron-transport layer by vapour deposition.

These processes are generally known to the person skilled in the art and can be applied by him without inventive step to organic electroluminescent devices comprising the compounds according to the invention.

The processing of the compounds according to the invention from the liquid phase, for example by spin coating or by printing processes, requires formulations of the compounds according to the invention. These formulations can be, for example, solutions, dispersions or mini-emulsions. It may be preferred to use mixtures of two or more solvents for this purpose. Suitable and preferred solvents are, for example, toluene, anisole, o-, m- or p-xylene, methyl benzoate, dimethylanisole, mesitylene, tetralin, veratrol, THF, methyl-THF, THP, chlorobenzene, dioxane or mixtures of these solvents.

The present invention therefore furthermore relates to a formulation, in particular a solution, dispersion or mini-emulsion, comprising at least one compound of the formula (1) or formula (2) or the preferred embodiments indicated above and at least one solvent, in particular an organic solvent. The way in which solutions of this type can be prepared is known to the person skilled in the art and is described, for example, in WO 2002/072714, WO 2003/019694 and the literature cited therein.

The present invention furthermore relates to mixtures comprising at least one compound of the formula (1) or formula (2) or the preferred embodiments indicated above and at least one further compound. The further compound can be, for example, a fluorescent or phosphorescent dopant if the compound according to the invention is used as matrix material. The mixture may then also additionally comprise a further material as additional matrix material.

The compounds according to the invention and the organic electroluminescent devices according to the invention are distinguished by the following surprising advantages over the prior art:
1. The compounds according to the invention are very highly suitable for use in a hole-blocking or electron-transport layer in an organic electroluminescent device. They are also suitable, in particular, for use in a hole-blocking layer which is directly adjacent to a phosphorescent emitting layer, since the compounds according to the invention do not extinguish the luminescence.
2. The compounds according to the invention, employed as matrix material for fluorescent or phosphorescent emitters, result in very high efficiencies and long lifetimes. This applies, in particular, if the compounds are employed as matrix material together with a further matrix material and a phosphorescent emitter.
3. The compounds according to the invention, employed in organic electroluminescent devices, result in high efficiencies and in steep current/voltage curves with low use and operating voltages.

These above-mentioned advantages are not accompanied by an impairment in the other electronic properties.

The invention is explained in greater detail by the following examples, without wishing to restrict it thereby. On the basis of the descriptions, the person skilled in the art will be able to carry out the invention throughout the range disclosed and prepare further compounds according to the invention without inventive step and use them in electronic devices or use the process according to the invention.

### EXAMPLES

The following syntheses are carried out, unless indicated otherwise, in dried solvents under a protective-gas atmosphere. The starting materials can be purchased from ALDRICH (potassium fluoride (spray-dried), tri-tert-butylphosphine, palladium(II) acetate). 3-Chloro-5,6-diphenyl-1,2,4-triazine is prepared analogously to EP 577559. 2′,7′-Di-tert-butylspiro-9,9′-bifluorene-2,7-bisboronic acid glycol ester is prepared in accordance with WO 2002/077060 and 2-chloro-4,6-diphenyl-1,3,5-triazine is prepared in accordance with U.S. Pat. No. 5,438,138. Spiro-9,9′-bifluorene-2,7-bis(boronic acid glycol ester) is prepared analogously to WO 2002/077060. The numbers in the case of the starting materials known from the literature, some of which are indicated in square brackets, are the corresponding CAS numbers.

### Example 1

### 1-Bromospiro-9,9′-bifluorene

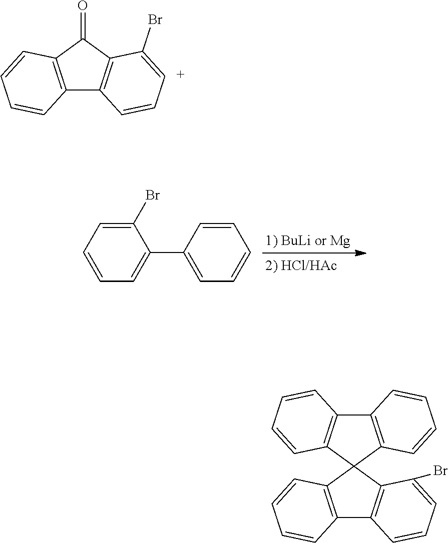 [SMILES: O=C1C2=C(C=C[C+]=C2Br)c2ccccc21 ; Brc1ccccc1-c1ccccc1 ; Brc1cccc2c1C1(c3ccccc3-c3ccccc31)c1ccccc1-2]

The corresponding Grignard reagent is prepared from 2.7 g (110 mmol) of iodine-activated magnesium turnings and a mixture of 25.6 g (110 mmol) of 2-bromobiphenyl, 0.8 ml of 1,2-dichloroethane, 50 ml of 1,2-dimethoxyethane, 400 ml of THF and 200 ml of toluene with secondary heating using an oil bath at 70° C. When the magnesium has reacted completely, the mixture is allowed to cool to room temperature, and a solution of 25.9 g (100 mmol) of 1-bromofluorenone[36804-63-4] in 500 ml of THF is then added dropwise, the reaction mixture is warmed at 50° C. for 4 h and then stirred at room temperature for a further 12 h. 100 ml of water are added, the mixture is stirred briefly, the organic phase is separated off, and the solvent is removed in vacuo. The residue is suspended in 500 ml of warm glacial acetic acid at 40° C., 0.5 ml of conc. sulfuric acid is added to the suspension, and the mixture is subsequently stirred at 100° C. for a further 2 h. After cooling, the precipitated solid is filtered off with suction, washed once with 100 ml of glacial acetic acid, three times with 100 ml of ethanol each time and finally recrystallised from dioxane. Yield: 26.9 g (68 mmol), 68%; purity about 98% according to ¹H-NMR.

The following compound is obtained analogously:

| | Starting | Starting | | |
|---|---|---|---|---|
| | material 1 | material 2 | Product | Yield |
| 1a | 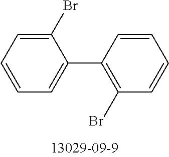 [SMILES: Brc1ccccc1-c1ccccc1Br] | 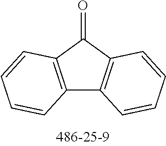 [SMILES: O=C1c2ccccc2-c2ccccc21] | 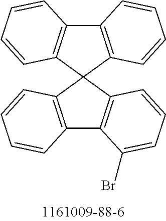 [SMILES: Brc1cccc2c1-c1ccccc1C21c2ccccc2-c2ccccc21] | 90% |

### Example 2

### Synthesis of 4-(4,6-diphenyl-1,3,5-triazin-2-yl)spiro-9,9′-bifluorene

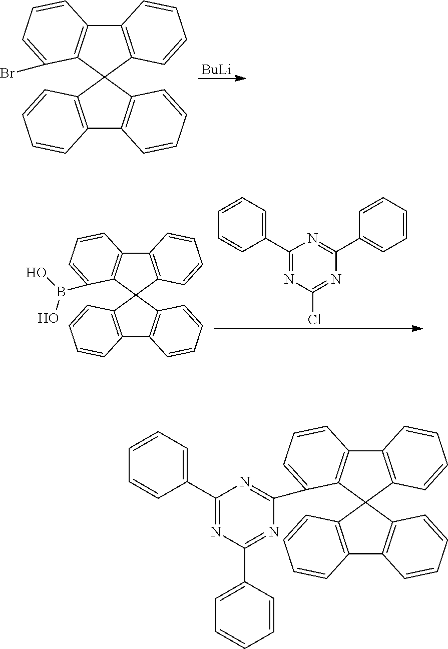

### Step 1) Synthesis of spiro-9,9′-bifluorene-1-boronic acid

110 ml (276 mmol) of n-buthyllithium (2.5 M in hexane) are added dropwise to a solution, cooled to −78° C., of 106 g (270 mmol) of 1-bromo-9-spirobifluorene in 1500 ml of diethyl ether. The reaction mixture is stirred at −78° C. for 30 min. The mixture is allowed to come to room temperature, re-cooled to −78° C., and a mixture of 40 ml (351 mmol) of trimethyl borate in 50 ml of diethyl ether is then added rapidly. After warming to −10° C., the mixture is hydrolysed using 135 ml of 2 N hydrochloric acid. The organic phase is separated off, washed with water, dried over sodium sulfate and evaporated to dryness. The residue is taken up in 300 ml of n-heptane, the colourless solid is filtered off with suction, washed with n-heptane and dried in vacuo. Yield: 94.5 g (255 mmol), 99% of theory; purity: 99% according to HPLC.

The following compound is obtained analogously:

| | Starting | | |
|---|---|---|---|
| | material 1 | Product | Yield |
| 2a | 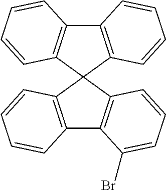 [SMILES: Brc1cccc2c1-c1ccccc1C21c2ccccc2-c2ccccc21] | 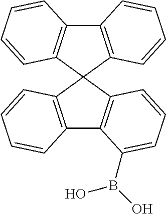 [SMILES: OB(O)c1cccc2c1-c1ccccc1C21c2ccccc2-c2ccccc21] | 83% |

### Step 2) Synthesis of 1-(4,6-diphenyl-1,3,5-triazin-2-yl)spiro-9,9′-bifluorene

56.8 g (110 mmol) of spiro-9,9′-bifluorene-1-boronic acid, 29.5 g (110.0 mmol) of 2-chloro-4,6-diphenyl-1,3,5-triazine and 44.6 g (210.0 mmol) of tripotassium phosphate are suspended in 500 ml of toluene, 500 ml of dioxane and 500 ml of water. 913 mg (3.0 mmol) of tri-o-tolylphosphine and then 112 mg (0.5 mmol) of palladium(II) acetate are added to this suspension, and the reaction mixture is heated under reflux for 16 h. After cooling, the organic phase is separated off, filtered through silica gel, washed three times with 200 ml of water and subsequently evaporated to dryness. The residue is recrystallised from toluene and from dichloromethane/isopropanol and finally sublimed in a high vacuum (p=5×10⁻⁵ mbar, T=377° C.). The yield is 38.7 g (43.5 mmol), corresponding to 87% of theory.

The following compounds are obtained analogously:

| | Starting | Starting | | |
|---|---|---|---|---|
| | material 1 | material 2 | Product | Yield |
| 2b | 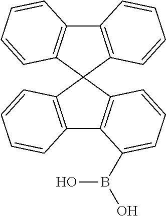 [SMILES: OB(O)c1cccc2c1-c1ccccc1C21c2ccccc2-c2ccccc21] | 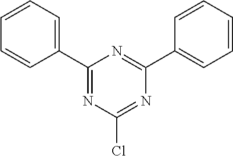 [SMILES: Clc1nc(-c2ccccc2)nc(-c2ccccc2)n1] 3842-55-5 | 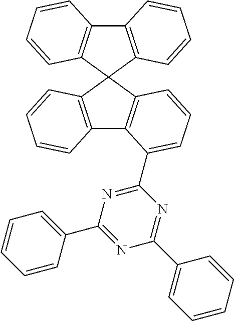 [SMILES: c1ccc(-c2nc(-c3ccccc3)nc(-c3cccc4c3-c3ccccc3C43c4ccccc4-c4ccccc43)n2)cc1] | 81% |
| 2c | 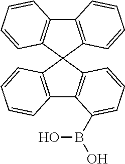 [SMILES: OB(O)c1cccc2c1-c1ccccc1C21c2ccccc2-c2ccccc21] | 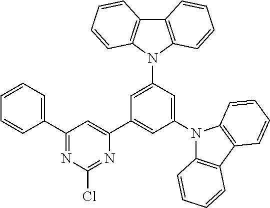 [SMILES: Clc1nc(-c2ccccc2)cc(-c2cc(-n3c4ccccc4c4ccccc43)cc(-n3c4ccccc4c4ccccc43)c2)n1] 1333505-18-2 | 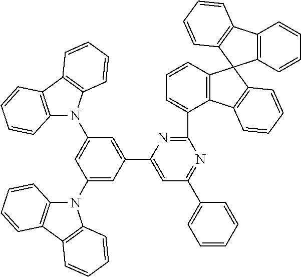 [SMILES: c1ccc(-c2cc(-c3cc(-n4c5ccccc5c5ccccc54)cc(-n4c5ccccc5c5ccccc54)c3)nc(-c3cccc4c3-c3ccccc3C43c4ccccc4-c4ccccc43)n2)cc1] | 80% |
| 2d | 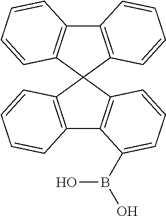 [SMILES: OB(O)c1cccc2c1-c1ccccc1C21c2ccccc2-c2ccccc21] | 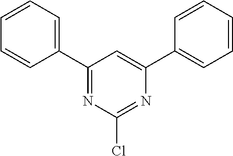 [SMILES: Clc1nc(-c2ccccc2)cc(-c2ccccc2)n1] 2915-16-4 | 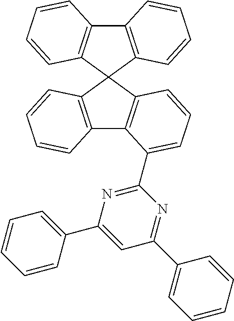 [SMILES: c1ccc(-c2cc(-c3ccccc3)nc(-c3cccc4c3-c3ccccc3C43c4ccccc4-c4ccccc43)n2)cc1] | 79% |
| 2e | 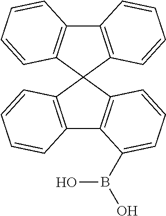 [SMILES: OB(O)c1cccc2c1-c1ccccc1C21c2ccccc2-c2ccccc21] | 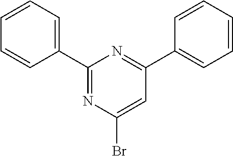 [SMILES: Brc1cc(-c2ccccc2)nc(-c2ccccc2)n1] 40734-4-5 | 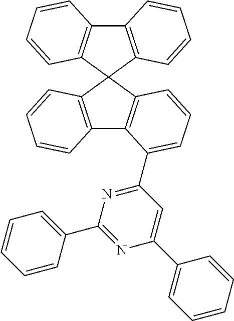 [SMILES: c1ccc(-c2cc(-c3cccc4c3-c3ccccc3C43c4ccccc4-c4ccccc43)nc(-c3ccccc3)n2)cc1] | 77% |
| 2f | 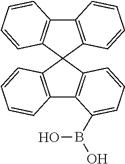 [SMILES: OB(O)c1cccc2c1-c1ccccc1C21c2ccccc2-c2ccccc21] | 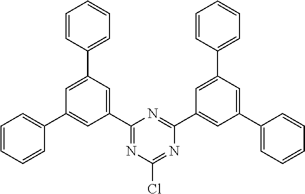 [SMILES: Clc1nc(-c2cc(-c3ccccc3)cc(-c3ccccc3)c2)nc(-c2cc(-c3ccccc3)cc(-c3ccccc3)c2)n1] 1205748-51-1 | 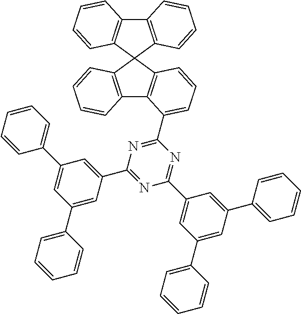 [SMILES: c1ccc(-c2cc(-c3ccccc3)cc(-c3nc(-c4cc(-c5ccccc5)cc(-c5ccccc5)c4)nc(-c4cccc5c4-c4ccccc4C54c5ccccc5-c5ccccc54)n3)c2)cc1] | 87% |
| 2g | 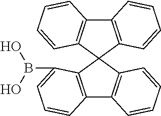 [SMILES: OB(O)c1cccc2c1C1(c3ccccc3-c3ccccc31)c1ccccc1-2] | 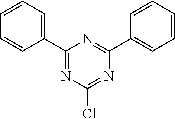 [SMILES: Clc1nc(-c2ccccc2)nc(-c2ccccc2)n1] 3842-55-5 | 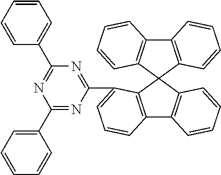 [SMILES: c1ccc(-c2nc(-c3ccccc3)nc(-c3cccc4c3C3(c5ccccc5-c5ccccc53)c3ccccc3-4)n2)cc1] | 76% |
| 2h | 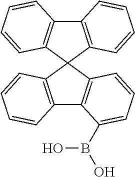 [SMILES: OB(O)c1cccc2c1-c1ccccc1C21c2ccccc2-c2ccccc21] | 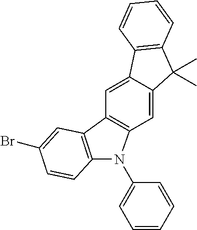 [SMILES: CC1(C)c2ccccc2-c2cc3c4cc(Br)ccc4n(-c4ccccc4)c3cc21] | 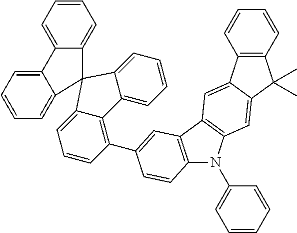 [SMILES: CC1(C)c2ccccc2-c2cc3c4cc(-c5cccc6c5-c5ccccc5C65c6ccccc6-c6ccccc65)ccc4n(-c4ccccc4)c3cc21] | 65% |
| | | 1257220-44-2 | | |
| 2i | 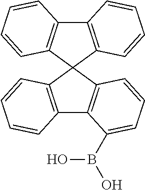 [SMILES: OB(O)c1cccc2c1-c1ccccc1C21c2ccccc2-c2ccccc21] | 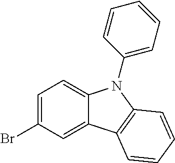 [SMILES: Brc1ccc2c(c1)c1ccccc1n2-c1ccccc1] 1153-85-1 | 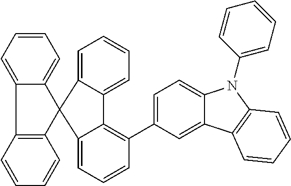 [SMILES: c1ccc(-n2c3ccccc3c3cc(-c4cccc5c4-c4ccccc4C54c5ccccc5-c5ccccc54)ccc32)cc1] | 79% |
| 2j | 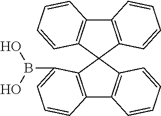 [SMILES: OB(O)c1cccc2c1C1(c3ccccc3-c3ccccc31)c1ccccc1-2] | 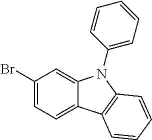 [SMILES: Brc1ccc2c3ccccc3n(-c3ccccc3)c2c1] | 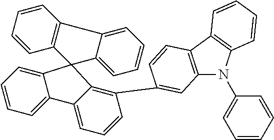 [SMILES: c1ccc(-n2c3ccccc3c3ccc(-c4cccc5c4C4(c6ccccc6-c6ccccc64)c4ccccc4-5)cc32)cc1] | 69% |
| | | 94994-62-4 | | |
| 2k | 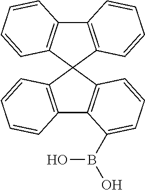 [SMILES: OB(O)c1cccc2c1-c1ccccc1C21c2ccccc2-c2ccccc21] | 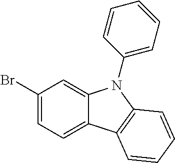 [SMILES: Brc1ccc2c3ccccc3n(-c3ccccc3)c2c1] 94994-62-4 | 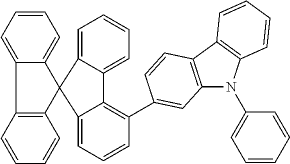 [SMILES: c1ccc(-n2c3ccccc3c3ccc(-c4cccc5c4-c4ccccc4C54c5ccccc5-c5ccccc54)cc32)cc1] | 73% |

### Example 3

### Production of the OLEDs

OLEDs according to the invention and OLEDs in accordance with the prior art are produced by a general process in accordance with WO 2004/058911, which is adapted to the circumstances described here (layer-thickness variation, materials).

The data of various OLEDs are presented in the following Examples V1 to E3 (see Tables 1 and 2). Glass plates coated with structured ITO (indium tin oxide) in a thickness of 50 nm are coated with 20 nm of PEDOT:PSS (poly(3,4-ethylenedioxythiophene) poly(styrenesulfonate), purchased as CLEVIOS™ P VP Al 4083 from Heraeus Precious Metals GmbH Germany, applied by spin coating from aqueous solution) for improved processing. These coated glass plates form the substrates to which the OLEDs are applied. The OLEDs have in principle the following layer structure: glass substrate/ITO/optional hole-injection layer (HIL)/hole-transport layer (HTL)/optional interlayer (IL)/electron-blocking layer (EBL)/emission layer (EML) optional hole-blocking layer (HBL)/electron-transport layer (ETL)/optional electron-injection layer (EIL) and finally a cathode. The cathode is formed by an aluminium layer with a thickness of 100 nm. The precise structure of the OLEDs is shown in Table 1. The other materials required for the production of the OLEDs are shown in Table 3.

All materials are applied by thermal vapour deposition in a vacuum chamber. The emission layer here always consists of at least one matrix material (also host material) and an emitting dopant (emitter), which is admixed with the matrix material or matrix materials in a certain proportion by volume by coevaporation. An expression such as Host1:Host3:TEG1 (30%:60%:10%) here means that material Host3 is present in the layer in a proportion by volume of 60%, Host1 is present in the layer in a proportion of 30% and TEG1 is present in the layer in a proportion of 10%. Analogously, the electron-transport layer may also consist of a mixture of two materials.

The OLEDs are characterised by standard methods. For this purpose, the electroluminescence spectra, the current efficiency (measured in cd/A), the power efficiency (measured in Im/W) and the external quantum efficiency (EQE, measured in per cent) as a function of the luminous density, calculated from current/voltage/luminous density characteristic lines (IUL characteristic lines) assuming Lambert emission characteristics, and the lifetime are determined. The electroluminescence spectra are determined at a luminous density of 1000 cd/m² and the CIE 1931 x and y colour coordinates are calculated therefrom. The term U1000 in Table 2 denotes the voltage required for a luminous density of 1000 cd/m². CE1000 and PE1000 denote the current and power efficiency respectively which are achieved at 1000 cd/m². Finally, EQE1000 denotes the external quantum efficiency at an operating luminous density of 1000 cd/m².

The data of the various OLEDs are summarised in Table 2. Examples V1 to V4 are comparative examples in accordance with the prior art, Examples E1 to E3 show data of OLEDs comprising materials according to the invention.

Some of the examples are explained in greater detail below in order to illustrate the advantages of the compounds according to the invention.

However, it should be pointed out that this only represents a selection of the data shown in Table 2.

### Use of Compounds According to the Invention in the Electron-Transport Layer

If compound Host2 is employed as electron-transport layer, a low operating voltage of 3.5 V and a very good quantum efficiency of almost 17% are obtained (Example E2), these values are worse on use of compound Host1 in accordance with the prior art (Example V2).

### Use of Compounds According to the Invention as Matrix Materials in Phosphorescent OLEDs

On use of materials according to the invention as single matrix material in combination with the green dopant TEG1, very low operating voltages of 4.4 V and very good quantum efficiencies of 15.6% are obtained (Example E1), whereas the voltage in Comparative Example V1 is higher for virtually the same efficiency.

Very good performance data are also obtained in a mixed-matrix system with materials according to the invention. In combination with material Host3, for example, a very low operating voltage of 3.6 V is obtained (Example E3), whereas this is significantly higher in Comparative Example V4.

TABLE 1
Structure of the OLEDs

| | HIL | HTL | IL | EBL | | HBL | | EIL |
|---|---|---|---|---|---|---|---|---|
| | Thick- | Thick- | Thick- | Thick- | EML | Thick- | ETL | Thick- |
| Ex. | ness | ness | ness | ness | Thickness | ness | Thickness | ness |
| V1 | — | HIM1 | HATCN | HTM1 | Host1:TEG1 | HBM1 | ETM1:LiQ | — |
| | | 70 nm | 5 nm | 90 nm | (90%:10%) 30 nm | 10 nm | (50%:50%) | |
| | | | | | | | 30 nm | |
| V2 | — | HIM1 | HATCN | HTM2 | HBM1:TEG1 | | Host1:LiQ | — |
| | | 70 nm | 5 nm | 90 nm | (90%:10%) 30 nm | | (50%:50%) | |
| | | | | | | | 30 nm | |
| V3 | — | HIM1 | HATCN | HTM1 | Host4:Host3:TEG1 | HBM1 | ETM1:LiQ | — |
| | | 70 nm | 5 nm | 90 nm | (30%:80%:10%) 30 nm | 10 nm | (50%:50%) | |
| | | | | | | | 30 nm | |
| V4 | — | HIM1 | HATCN | HTM1 | Host1:Host3:TEG1 | HBM1 | ETM1:LiQ | — |
| | | 70 nm | 5 nm | 90 nm | (30%:60%:10%) 30 nm | 10 nm | (50%:50%) | |
| | | | | | | | 30 nm | |
| E1 | — | HIM1 | HATCN | HTM1 | Host2:TEG1 | HBM1 | ETM1:LiQ | — |
| | | 70 nm | 5 nm | 90 nm | (90%:10%) 30 nm | 10 nm | (50%: 50%) | |
| | | | | | | | 30 nm | |
| E2 | — | HIM1 | HATCN | HTM2 | HBM1:TEG1 | | Host2:LiQ | — |
| | | 70 nm | 5 nm | 90 nm | (90%:10%) 30 nm | | (50%:50%) | |
| | | | | | | | 30 nm | |
| E3 | — | HIM1 | HATCN | HTM1 | Host2:Host3:TEG1 | HBM1 | ETM1:LiQ | — |
| | | 70 nm | 5 nm | 90 nm | (30%:60%:10%) 30 nm | 10 nm | (50%:50%) | |
| | | | | | | | 30 nm | |

TABLE 2
Data of the OLEDs

| | U1000 | CE1000 | PE1000 | EQE1000 | CIE x/y at |
|---|---|---|---|---|---|
| Ex. | (V) | (cd/A) | (lm/W) | (%) | 1000 cd/M1 |
| V1 | 4.8 | 55 | 36 | 15.9 | 0.39/0.58 |
| V2 | 3.8 | 56 | 46 | 16.0 | 0.38/0.59 |
| V3 | 4.0 | 52 | 41 | 14.7 | 0.39/0.58 |
| V4 | 3.9 | 53 | 43 | 15.0 | 0.39/0.58 |
| E1 | 4.4 | 54 | 39 | 15.6 | 0.39/0.58 |
| E2 | 3.5 | 59 | 53 | 16.9 | 0.38/0.59 |
| E3 | 3.6 | 54 | 46.5 | 15.0 | 0.38/0.59 |

TABLE 3
Structural formulae of the materials for the OLEDs

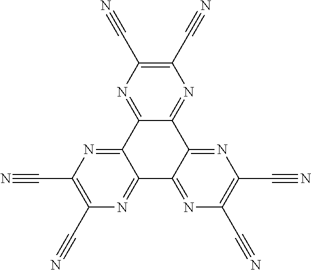 [SMILES: N#Cc1nc2c3nc(C#N)c(C#N)nc3c3nc(C#N)c(C#N)nc3c2nc1C#N]

HATCN

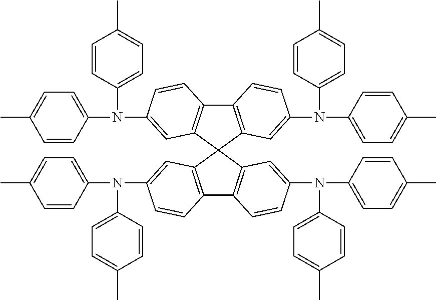

HIM1

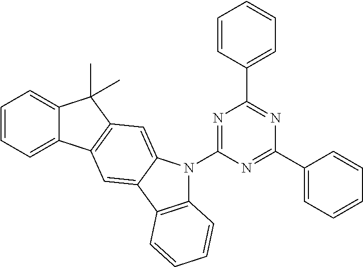 [SMILES: CC1(C)c2ccccc2-c2cc3c4ccccc4n(-c4nc(-c5ccccc5)nc(-c5ccccc5)n4)c3cc21]

HBM1

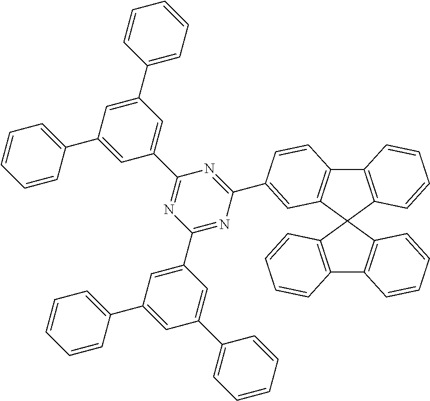 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1]

ETM1 (prior art)

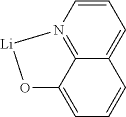

LiQ

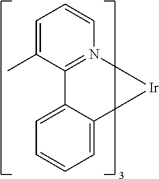

TEG1

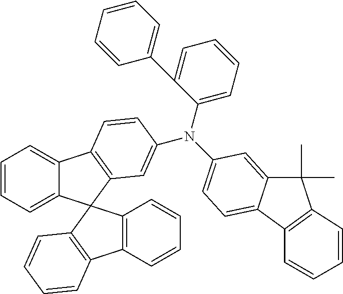 [SMILES: CC1(C)c2ccccc2-c2ccc(N(c3ccc4c(c3)C3(c5ccccc5-c5ccccc53)c3ccccc3-4)c3ccccc3-c3ccccc3)cc21]

HTM1

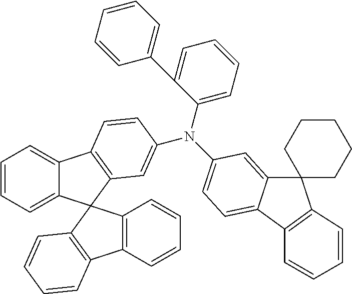 [SMILES: c1ccc(-c2ccccc2N(c2ccc3c(c2)C2(CCCCC2)c2ccccc2-3)c2ccc3c(c2)C2(c4ccccc4-c4ccccc42)c2ccccc2-3)cc1]

HTM2

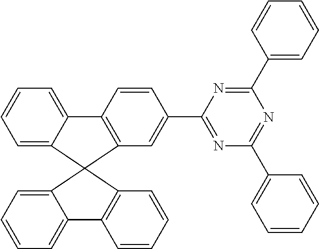 [SMILES: c1ccc(-c2nc(-c3ccccc3)nc(-c3ccc4c(c3)C3(c5ccccc5-c5ccccc53)c3ccccc3-4)n2)cc1]

Host 1 (prior art)

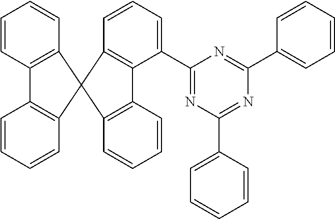 [SMILES: c1ccc(-c2nc(-c3ccccc3)nc(-c3cccc4c3-c3ccccc3C43c4ccccc4-c4ccccc43)n2)cc1]

Host 2 (according to the invention)

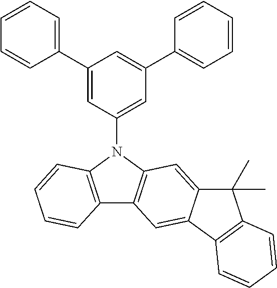 [SMILES: CC1(C)c2ccccc2-c2cc3c4ccccc4n(-c4cc(-c5ccccc5)cc(-c5ccccc5)c4)c3cc21]

Host 3

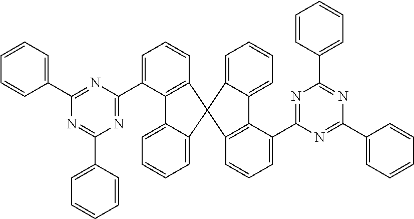 [SMILES: c1ccc(-c2nc(-c3ccccc3)nc(-c3cccc4c3-c3ccccc3C43c4ccccc4-c4c(-c5nc(-c6ccccc6)nc(-c6ccccc6)n5)cccc43)n2)cc1]

Host 4 (prior art)

1-15. (canceled)

16. A compound of the formula (1) or (2), 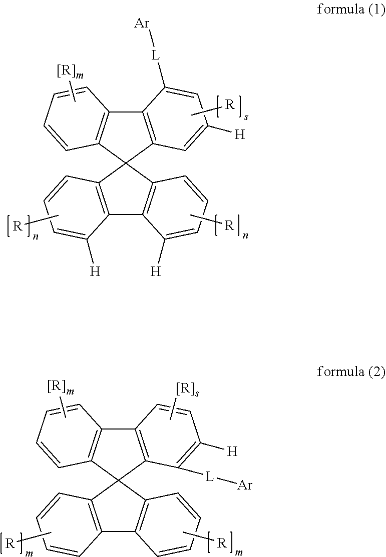
where the following applies to the symbols and indices used:
Ar is a heteroaromatic ring system having 5 to 40 aromatic ring atoms which is bonded to L via a carbon atom if L stands for a single bond, and which is bonded to L via a carbon atom or a nitrogen atom if L is not equal to a single bond, and which may be substituted by one or more radicals R¹; or Ar is an aromatic ring system having 6 to 40 aromatic ring atoms, which may be substituted by one or more radicals R¹, if L stands for C(═O);
L is a single bond, C(═O) or an aromatic ring system having 5 to 24 aromatic ring atoms, which may be substituted by one or more radicals R;
R, R¹ is selected on each occurrence, identically or differently, from the group consisting of H, D, F, Cl, Br, I, CN, Si(R²)₃, a straight-chain alkyl, alkoxy or thioalkyl group having 1 to 40 C atoms or a branched or cyclic alkyl, alkoxy or thioalkyl group having 3 to 40 C atoms, each of which may be substituted by one or more radicals R², where in each case one or more non-adjacent CH₂ groups may be replaced by Si(R²)₂, C═NR², P(═O)(R²), SO, SO₂, NR², O, S or CONR² and where one or more H atoms may be replaced by D, F, Cl, Br or I, an aromatic or heteroaromatic ring system having 6 to 40 C atoms, which may in each case be substituted by one or more radicals R², an aryloxy group having 5 to 60 aromatic ring atoms, which may be substituted by one or more radicals R², or an aralkyl group having 5 to 60 aromatic ring atoms, which may in each case be substituted by one or more radicals R², where two or more adjacent substituents R or R¹ may optionally form a mono- or polycyclic, aliphatic ring system, which may be substituted by one or more radicals R²;
R² is selected from the group consisting of H, D, F, an aliphatic hydrocarbon radical having 1 to 20 C atoms or an aromatic or heteroaromatic ring system having 5 to 30 C atoms, in which one or more H atoms may be replaced by D or F, where two or more adjacent substituents R² may form a mono- or polycyclic, aliphatic, ring system with one another;
s is 0, 1 or 2;
m is on each occurrence, identically or differently, 0, 1, 2, 3 or 4;
n is on each occurrence, identically or differently, 0, 1, 2 or 3.

17. The compound according to claim 16, wherein the compound is a compound of the formula (1a) or formula (2a), 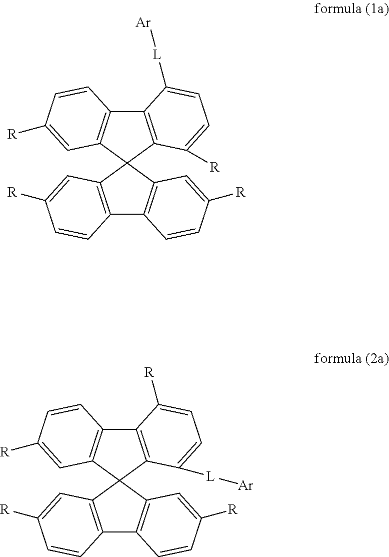
where the symbols used have the meanings given in claim 16.

18. The compound according to claim 16, wherein the compound is a compound of the formula (1b) or (2b), 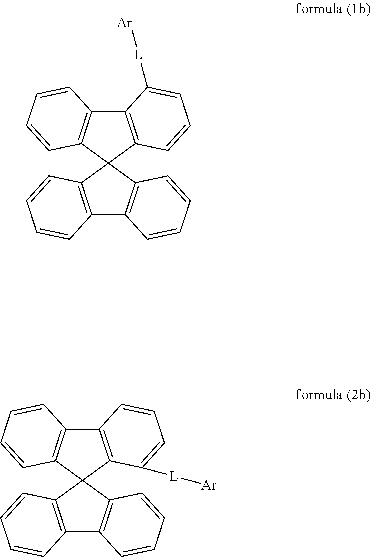
where the symbols used have the meanings given in claim 16.

19. The compound according to claim 16, wherein L is selected from a single bond, C(═O) or an aromatic ring system having 6 to 12 aromatic ring atoms, which may be substituted by one or more radicals R.

20. The compound according to claim 16, wherein L is selected from the group consisting of a single bond or an ortho-, meta- or para-linked phenylene group, which may be substituted by one or more radicals R, but is preferably unsubstituted.

21. The compound according to claim 16, wherein Ar represents a heteroaromatic ring system having 5 to 24 aromatic ring atoms which may in each case be substituted by one or more radicals R¹; or in that Ar represents an aromatic ring system having 6 to 24 aromatic ring atoms, which may be substituted by one or more radicals R¹, if L stands for C(═O).

22. The compound according to claim 16, wherein Ar represents a heteroaromatic ring system having 5 to 13 aromatic ring atoms, which may in each case be substituted by one or more radicals R1; or in that Ar represents an aromatic ring system having 6 to 24 aromatic ring atoms, which may be substituted by one or more radicals R1, if L stands for C(═O).

23. The compound according to claim 16, wherein Ar is selected from the group consisting of triazine, pyrimidine, pyrazine, pyridazine, pyridine, pyrazole, imidazole, oxazole, oxadiazole, thiazole, benzimidazole, benzofuran, benzothiophene, indole, dibenzofuran, dibenzothiophene, carbazole, indenocarbazole and indolocarbazole, where these groups may each be substituted by one or more radicals R¹; or in that Ar, if L stands for C(═O), is selected from the group consisting of phenyl, biphenyl, ortho-, meta- or para-terphenyl, ortho-, meta-, para- or branched quaterphenyl, 1-, 2-, 3- or 4-fluorenyl or 1-, 2-, 3- or 4-spirobifluorenyl, each of which may be substituted by one or more radicals R¹.

24. The compound according to claim 16, wherein Ar selected from the structures of the formulae (Ar-1) to (Ar-24), 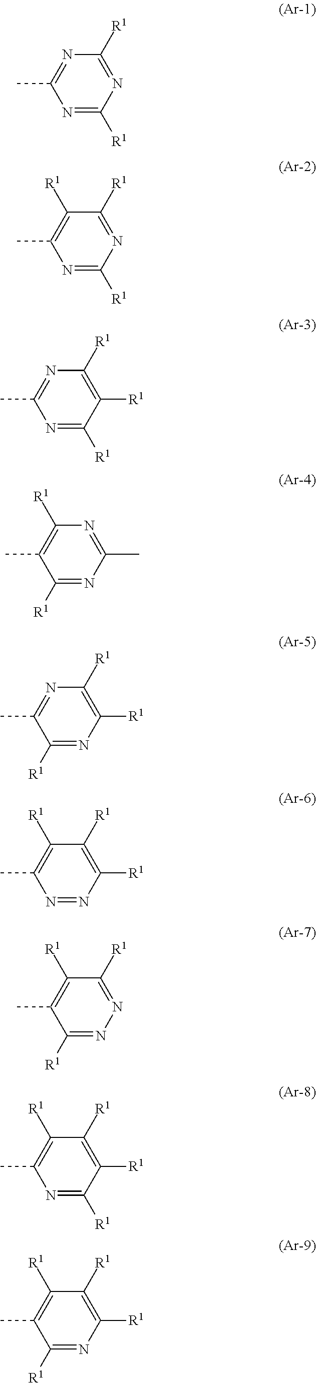 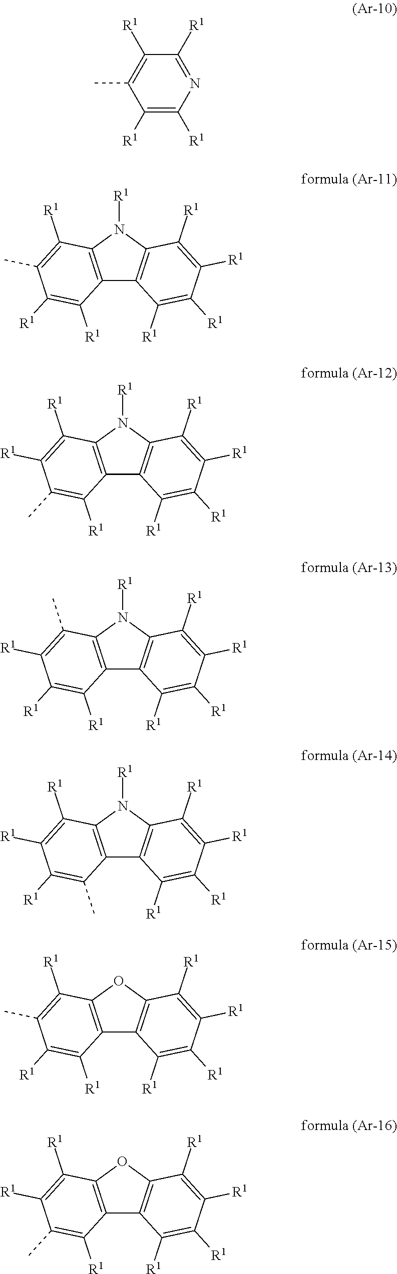 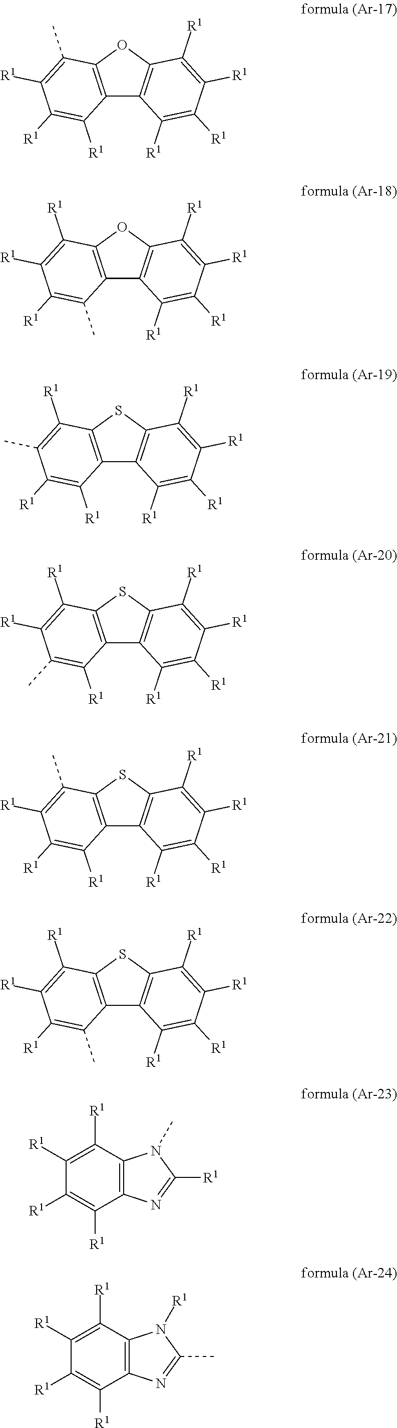
where the dashed bond indicates the bond to L and R¹ has the meanings given in claim 16.

25. The compound according to claim 16, wherein R and R¹ are selected, identically or differently on each occurrence, from the group consisting of H, D, F, CN, a straight-chain alkyl or alkoxy group having 1 to 10 C atoms or a branched or cyclic alkyl or alkoxy group having 3 to 10 C atoms, each of which may be substituted by one or more radicals R², where one or more non-adjacent CH₂ groups may be replaced by O and where one or more H atoms may be replaced by F, or an aromatic or heteroaromatic ring system having 6 to 24 aromatic ring atoms, which may in each case be substituted by one or more radicals R².

26. The compound according to claim 16, wherein the following applies to the symbols used:
L is a single bond, C(═O) or an aromatic ring system having 6 to 12 aromatic ring atoms, which may be substituted by one or more radicals R;
Ar is a heteroaromatic ring system having 5 to 13 aromatic ring atoms, which may be substituted by one or more radicals R¹, where Ar is bonded to L via a carbon atom if L stands for a single bond, or is bonded to L via a carbon or nitrogen atom if L is not equal to a single bond; or is an aromatic ring system having 6 to 24 aromatic ring atoms, which may be substituted by one or more radicals R1, if L stands for C(═O);
R, R¹ is selected, identically or differently on each occurrence, from the group consisting of H, D, F, CN, a straight-chain alkyl or alkoxy group having 1 to 10 C atoms or a branched or cyclic alkyl or alkoxy group having 3 to 10 C atoms, each of which may be substituted by one or more radicals R², where one or more non-adjacent CH₂ groups may be replaced by O and where one or more H atoms may be replaced by F, an aromatic or heteroaromatic ring system having 6 to 24 aromatic ring atoms, which may in each case be substituted by one or more radicals R².

27. A process for the preparation of the compound according to claim 16, wherein the group -L-Ar is introduced by a metal-catalysed coupling reaction between a 1- or 4-functionalised spirobifluorene and a functionalised group -L-Ar.

28. A formulation comprising at least one compound according to claim 16.

29. A solution, dispersion or miniemulsion comprising at least one compound according to claim 16 and at least one solvent,

30. A mixture comprising at least one compound according to claim 16 and at least one fluorescent or phosphorescent compound.

31. An organic electroluminescent device which comprises the compound according to claim 16.

32. An electronic device comprising at least one compound according to claim 16.

33. The electronic device as claimed in claim 32, wherein the electronic device is selected from the group consisting of organic electroluminescent devices, organic integrated circuits, organic field-effect transistors, organic thin-film transistors, organic light-emitting transistors, organic solar cells, dye-sensitised organic solar cells, organic optical detectors, organic photoreceptors, organic field-quench devices, light-emitting electrochemical cells, organic laser diodes and organic plasmon emitting devices.

34. An organic electroluminescent device which comprises the compound according to claim 16 is employed as electron-transport material in an electron-transport or hole-blocking layer or as matrix material for fluorescent or phosphorescent emitters in an emitting layer.
